SEQUENCE LISTING (42 sequences)

SEQ ID NO: 1 - DNA, 20 bp, Homo sapiens
  source 1..20: mol_type = other DNA
gccatatcctgaagtgtccg

SEQ ID NO: 2 - DNA, 20 bp, Homo sapiens
  source 1..20: mol_type = other DNA
tctggccatatcctgaagtg

SEQ ID NO: 3 - DNA, 20 bp, Homo sapiens
  source 1..20: mol_type = other DNA
tgctctggccatatcctgaa

SEQ ID NO: 4 - DNA, 20 bp, Homo sapiens
  source 1..20: mol_type = other DNA
gccatagcctgaagtgtccg

SEQ ID NO: 5 - DNA, 20 bp, Homo sapiens
  source 1..20: mol_type = other DNA
tctggccatagcctgaagtg

SEQ ID NO: 6 - DNA, 20 bp, Homo sapiens
  source 1..20: mol_type = other DNA
tgctctggccatagcctgaa

SEQ ID NO: 7 - DNA, 20 bp, Homo sapiens
  source 1..20: mol_type = other DNA
gccagggtaggaggactgct

SEQ ID NO: 8 - DNA, 20 bp, Homo sapiens
  source 1..20: mol_type = other DNA
catagccagggtaggaggac

SEQ ID NO: 9 - DNA, 20 bp, Homo sapiens
  source 1..20: mol_type = other DNA
ggccatagccagggtaggag

SEQ ID NO: 10 - DNA, 20 bp, Homo sapiens
  source 1..20: mol_type = other DNA
gccaggataggaggactgct

SEQ ID NO: 11 - DNA, 20 bp, Homo sapiens
  source 1..20: mol_type = other DNA
catagccaggataggaggac

SEQ ID NO: 12 - DNA, 20 bp, Homo sapiens
  source 1..20: mol_type = other DNA
ggccatagccaggataggag

SEQ ID NO: 13 - DNA, 20 bp, Homo sapiens
  source 1..20: mol_type = other DNA
tgacctcaagccctctgagt

SEQ ID NO: 14 - DNA, 20 bp, Homo sapiens
  source 1..20: mol_type = other DNA
tcaatgacctcaagccctct

SEQ ID NO: 15 - DNA, 20 bp, Homo sapiens
  source 1..20: mol_type = other DNA
atgtcaatgacctcaagccc

SEQ ID NO: 16 - DNA, 20 bp, Homo sapiens
  source 1..20: mol_type = other DNA
tgacctgaagccctctgagt

SEQ ID NO: 17 - DNA, 20 bp, Homo sapiens
  source 1..20: mol_type = other DNA
tcaatgacctgaagccctct

SEQ ID NO: 18 - skipped

SEQ ID NO: 19 - DNA, 20 bp, Homo sapiens
  source 1..20: mol_type = other DNA
catagccagagtaggaggac

SEQ ID NO: 20 - DNA, 20 bp, Homo sapiens
  source 1..20: mol_type = other DNA
catagccagtgtaggaggac

SEQ ID NO: 21 - DNA, 20 bp, Homo sapiens
  source 1..20: mol_type = other DNA
catagccaaggtaggaggac

SEQ ID NO: 22 - DNA, 20 bp, Homo sapiens
  source 1..20: mol_type = other DNA
catagccatggtaggaggac

SEQ ID NO: 23 - DNA, 20 bp, Homo sapiens
  source 1..20: mol_type = other DNA
catagccagggtaagaggac

SEQ ID NO: 24 - DNA, 20 bp, Homo sapiens
  source 1..20: mol_type = other DNA
catagccagggtatgaggac

SEQ ID NO: 25 - DNA, 20 bp, Homo sapiens
  source 1..20: mol_type = other DNA
catagcgagggtaggaggac

SEQ ID NO: 26 - DNA, 20 bp, Homo sapiens
  source 1..20: mol_type = other DNA
catagcaagggtaggaggac

SEQ ID NO: 27 - DNA, 20 bp, Homo sapiens
  source 1..20: mol_type = other DNA
catagccagaataggaggac

SEQ ID NO: 28 - DNA, 20 bp, Homo sapiens
  source 1..20: mol_type = other DNA
catagccagtataggaggac

SEQ ID NO: 29 - DNA, 20 bp, Homo sapiens
  source 1..20: mol_type = other DNA
catagccaagataggaggac

SEQ ID NO: 30 - DNA, 20 bp, Homo sapiens
  source 1..20: mol_type = other DNA
catagccatgataggaggac

SEQ ID NO: 31 - DNA, 20 bp, Homo sapiens
  source 1..20: mol_type = other DNA
catagccaggataagaggac

SEQ ID NO: 32 - DNA, 20 bp, Homo sapiens
  source 1..20: mol_type = other DNA
catagccaggatatgaggac

SEQ ID NO: 33 - DNA, 20 bp, Homo sapiens
  source 1..20: mol_type = other DNA
catagcgaggataggaggac

SEQ ID NO: 34 - DNA, 20 bp, Homo sapiens
  source 1..20: mol_type = other DNA
catagcaaggataggaggac

SEQ ID NO: 35 - DNA, 20 bp, Homo sapiens
  source 1..20: mol_type = other DNA
accttatccaatagcgcctc

SEQ ID NO: 36 - DNA, 20 bp, Homo sapiens
  source 1..20: mol_type = other DNA
ccactgtaactctgctgtcc

SEQ ID NO: 37 - DNA, 20 bp, Homo sapiens
  source 1..20: mol_type = other DNA
gtactcagcggtgttggaac

SEQ ID NO: 38 - DNA, 20 bp, Homo sapiens
  source 1..20: mol_type = other DNA
ccactactcatggaggattg

SEQ ID NO: 39 - DNA, 20 bp, Homo sapiens
  source 1..20: mol_type = other DNA
agcgcaagggtttctggttt

SEQ ID NO: 40 - DNA, 20 bp, Homo sapiens
  source 1..20: mol_type = other DNA
ggagtcatgggggagggata

SEQ ID NO: 41 - DNA, 21 bp, Homo sapiens
  source 1..21: mol_type = other DNA
ctatggaactcagtcaactcc

SEQ ID NO: 42 - DNA, 20 bp, Homo sapiens
  source 1..20: mol_type = other DNA
gaactgctaccgtaacttcc

(19) United States
(12) Patent Application Publication
(10) Pub. No.: US 2025/0382612 A1
(43) Pub. Date: Dec. 18, 2025

(54) COMPOSITIONS AND METHODS FOR TREATING DISEASES ASSOCIATED WITH PATHOGENIC FUS VARIANTS

(71) Applicant: THE PERRON INSTITUTE FOR NEUROLOGICAL AND TRANSLATIONAL SCIENCE LIMITED, Nedlands (AU)

(72) Inventors: Rita Mejzini, Yangebup (AU); Patrick Anthony Akkari, Mullaloo (AU); Loren Louise Flynn, Karrinyup (AU); Stephen Wilton, Applecross (AU); Susan Fletcher, Bayswater (AU)

(21) Appl. No.: 18/835,212

(22) PCT Filed: Feb. 2, 2023
(86) PCT No.: PCT/AU2023/050067

§ 371 Date: Aug. 1, 2024

(30) Foreign Application Priority Data
Feb. 2, 2022 (AU) 2022900200

(51) Int. Cl. C12N 15/113

(52) U.S. Cl. CPC C12N 15/113; C12N 2310/11; C12N 2310/314; C12N 2310/315; C12N 2310/321; C12N 2310/3233; C12N 2310/341; C12N 2310/346; C12N 2310/3513; C12N 2320/34

(57) ABSTRACT

The present invention relates to the field of antisense oligonucleotides used to reduce expression of selected alleles of the FUS gene which encodes the protein FUS or variants thereof. The invention also provides pharmaceutical compositions and methods to treat the effects of a disease associated with pathogenic FUS genetic variants by administration of antisense oligonucleotides and therapeutic compositions comprising AONs targeted to FUS or variants thereof.

### TECHNICAL FIELD

The present invention relates to antisense oligonucleotides (AONs) to reduce expression of the FUS gene which encodes FUS RNA binding protein (also known as Fused in Sarcoma). The invention provides methods to selectively reduce expression of specific alleles of the FUS transcript by administration of AONs in order to reduce expression of FUS that contains pathogenic variations associated with neurodegenerative disease.

This application contains, as a separate part of disclosure, a Sequence Listing in computer-readable form (Filename: 70698_SubSeqListing.xml; Size: 37,093 bytes; Creation Date: Mar. 12, 2025) which is incorporated by reference herein in its entirety.

### BACKGROUND ART

The following discussion of the background art is intended to facilitate an understanding of the present invention only. The discussion is not an acknowledgement or admission that any of the material referred to is or was part of the common general knowledge as at the priority date of the application.

### FUS

FUS encodes a ubiquitously expressed 526 amino acid protein belonging to the FET family of RNA binding proteins. FUS is predominantly localized to the nucleus under normal physiological conditions but crosses over to the cytoplasm, functioning in nucleocytoplasmic transport. FUS functions in a diverse range of cellular processes including transcription, pre-mRNA splicing, RNA transport and translation regulation. FUS is also involved in DNA repair mechanisms including both homologous recombination during DNA double-strand break repair and in non-homologous end joining. Additionally, FUS plays a role in the formation of paraspeckles providing cellular defence against various types of stress.

Up to 10% of Amyotrophic lateral sclerosis (ALS) affected individuals have at least one other affected family member and are defined as having familial ALS (fALS); almost all of these cases have been found to be inherited in an autosomal dominant manner. The remaining 90 to 95% of ALS cases occur in people with no prior family history; these individuals are said to have sporadic ALS (sALS). Pathogenic variations in FUS are responsible for approximately 5% of fALS cases and less than 1% of sALS cases.

Over 50 autosomal dominant FUS variants have now been identified in ALS patients. The majority are missense mutations, although in rare cases insertions, deletions, splicing and nonsense mutations have been reported. Many of the pathogenic variants are clustered within the nuclear localization signal and lead to the redistribution of FUS to the cytoplasm. Others occur in the glycine and arginine-rich regions, the prion-like domain and the 3′UTR. Variants within some regions appear to increase the propensity of the protein to form solid aggregates, pointing to various pathomechanisms operating in FUS related ALS.

FUS is autoregulated with one mechanism involving the protein binding to its own pre-mRNA to repress the expression of exon 7. A frameshift in exon-7 skipped splice variants results in a premature stop codon with the transcripts subject to nonsense mediated decay. In FUS-ALS (FUS-amyotrophic lateral sclerosis) and FUS-FTD (FUS-frontotemporal dementia), the mislocalisation of FUS to the cytoplasm may compromise autoregulation which could result in overexpression.

Impaired cellular function can also be the direct result of pathogenic FUS variants that have been reported to cause splicing defects, DNA damage and to compromise FUS autoregulation. Additionally, there are indications of a propagating mechanism of disease in FUS-ALS, possibly mediated by its prion-like protein domain.

Debate continues on the extent to which a loss of function or a gain of function mechanism causes disease in FUS-ALS. FUS loss of function theories suppose that the pathologic cytoplasmic redistribution of FUS renders it incapable of carrying out its functions in the nucleus. Evidence from mouse models have suggested that loss of FUS is not sufficient to cause ALS. However, the findings were contradictory when Drosophila FUS knockdown models were used, whereby neuronal degeneration and locomotive defects followed the knockdown of the FUS orthologue Cabeza.

There is strong evidence for gain of function mechanisms operating in FUS-ALS. A transgenic mouse model overexpressing wild-type human FUS reportedly developed an aggressive phenotype of motor neurodegeneration and evidence of cytoplasmic FUS accumulation. There is debate as to whether toxicity is primarily mediated by the FUS aggregates directly or via an increase in soluble FUS in the cytoplasm after its redistribution. Cytoplasmic FUS distribution also alters stress granule dynamics. Rather than purely pathologic, the propensity of FUS to aggregate is important in normal cellular functions. Some have proposed that FUS aggregation may be a compensatory mechanism protecting cells from potentially toxic increases in soluble cytoplasmic FUS.

Pathogenic FUS variants are associated with early onset and juvenile ALS which presents as a relentlessly progressive muscle atrophy and weakness, with the effects on respiratory muscles limiting survival to less than 3 years after disease onset in most cases. Current treatment options are based on symptom management and respiratory support with the only approved medications prolonging survival for just a few months or providing only modest benefits in some patients. Effective treatments that slow or pause disease progression are lacking.

Due to the strong evidence of a toxic gain of function caused by FUS aggregation, overexpression or cytoplasmic mislocalisation, knockdown of FUS is generally considered to be an acceptable therapeutic strategy for treating patients with pathogenic FUS mutations. Several patients received an investigational FUS targeted AON under the grounds of compassionate use with the treatment (ION363) now undergoing a phase 3 clinical trial (NCT04768972). As FUS plays key roles in several DNA repair mechanisms, is involved in the regulation and splicing of many other genes and plays a role in defence against cellular stress, there is concern that knocking down FUS could lead to health risks in the future due to its inability to perform these roles. For this reason, a strategy that to selectively knock down one FUS allele over another that can be targeted to the allele a patients pathogenic variation falls on has been pursued.

### Allele Selective Knockdown Strategy

FUS-ALS is a dominant disease, with the vast majority of affected patients carrying a deleterious variant in only one of their two alleles. This makes an AON based, allele specific approach to FUS knockdown a viable strategy to treat these patients. As FUS is autoregulated by its protein level, it may be possible to selectively knock down expression of the pre-mRNA and mRNA containing the pathogenic variant utilising AONs whilst leaving enough of the normal mRNA present to produce a physiologically healthy amount of protein.

This allele selective strategy uses a gapmer based AON design that takes advantage of the endogenous enzyme RNase H. RNase H is a non-sequence-specific endonuclease that catalyses the cleavage of the RNA strand in a DNA/RNA heteroduplex. Gapmers consist of a DNA based core that is compatible with RNase H activity flanked by a number of bases (wings) that can be extensively modified and do not need to be compatible with RNase H activity. Gapmers can be targeted directly to an area of RNA where there is variation between alleles and utilise the variation to produce selectivity to the target allele. Several properties of the gapmers, that may increase selectivity to the target allele can be modified with the effect generally needing to be determined experimentally. Modifications can include the number, placement and base composition of mismatches between the gapmer and the target RNA, the window (gap) or wing length as well as changes in molecule length and chemistry.

FUS-ALS is relatively rare and at least 50 autosomal dominant ALS-associated mutations in FUS have been reported. Given the large number of pathogenic variants, each variant is exceedingly rare, making it economically unviable for pharmaceutical companies to invest the resources into developing allele specific treatments for each, for this reason common variants in FUS have been targeted.

### FUS Proteinopathy

Reducing expression of FUS alleles that contain pathogenic variations has application in the prevention and treatment of diseases associated with FUS proteinopathy including in FUS-ALS and FUS-FTLD or other neurological conditions in which patients may have a pathogenic FUS mutation. It is also possible that expression of FUS alleles that contain pathogenic variations has application in the prevention and treatment of the following diseases: amyotrophic lateral sclerosis (ALS), frontotemporal dementia (FTD) and essential tremor (ET).

It is in the light of this background that the present invention has been developed. Particularly, the present invention seeks to provide a means for ameliorating FUS proteinopathy in diseases associated with pathogenic FUS genetic variants.

### SUMMARY OF THE INVENTION

The present invention is directed to compounds, particularly AONs, which are targeted to a nucleic acid encoding FUS. Embodiments of the present invention relate to AONs that are capable of binding to FUS pre-mRNA or mRNA.

Broadly, according to the first aspect of the invention, there is provided an antisense oligonucleotide targeted to a nucleic acid molecule encoding FUS pre-mRNA or mRNA, wherein the antisense oligonucleotide has a nucleobase sequence that is: (a) selected from the list consisting of: SEQ ID NO: 1 to SEQ ID NO: 34 or a variant thereof; or (b) complementary to at least 1 or more contiguous nucleobases in a target FUS pre-mRNA or mRNA to which SEQ ID NO: 1 to SEQ ID NO: 34 also binds or a variant thereof, wherein the antisense oligonucleotide inhibits the expression of at least one allele of the FUS gene or a FUS gene variant thereof and wherein the antisense oligonucleotide is substantially isolated or purified.

In one embodiment, the allele comprises a pathogenic variant of the FUS gene. In another embodiment, the pathogenic variant is a genetic alteration that increases the subject's susceptibility or predisposition to a disease.

In another embodiment, the disease is selected from the group consisting of: ALS, FTD, ET. Preferably, the disease is selected from the group consisting of: ALS or FTD. Preferably, the disease is selected from the group consisting of: FUS-ALS or FUS-FTD.

In another embodiment, the antisense oligonucleotide binds to an area of the transcript comprised within exon 3, exon 4 or the 3′ UTR on FUS. Preferably, the antisense oligonucleotide binds to an area of the transcript comprising CV (rs741810), CV2 (rs1052352), or CV3 (rs4889537).

In another embodiment, the antisense oligonucleotide selectively knocks down expression of one allele of the pre-mRNA and/or mRNA of FUS or a FUS variant thereof whilst leaving enough of the normal mRNA present to produce a substantial amount of protein.

In another embodiment, the antisense oligonucleotide induces RNase H mediated degradation of the FUS pre-mRNA and/or FUS mRNA.

In another embodiment, the antisense oligonucleotide is a thiophosphoramidate morpholino oligomer (TMO) chimera.

In another embodiment, the antisense oligonucleotide is a thiophosphoramidate morpholino oligomer (TMO) chimera with a gapmer design comprising TMO and phosphorothioate DNA or DNA subunits.

In another embodiment, the antisense oligonucleotide is a peptide-

thiophosphoramidate morpholino oligomer (TMO) chimera conjugate.

In another embodiment, the antisense oligonucleotide is selected from the list consisting of: SEQ ID NO: 1 to 12. Preferably, the antisense oligonucleotide is SEQ ID NO: 2, 4, 5, 8, or 11.

According to another aspect of the invention, there is provided a method of inducing selective knockdown of selected alleles of FUS pre-mRNA and mRNA, the method comprising the steps of: (a) providing one or more of the antisense oligonucleotides according to any one of claims 1 to 14; and (b) allowing the oligomer(s) to bind to a target nucleic acid site.

According to another aspect of the invention, there is provided composition to treat, prevent or ameliorate the effects of a disease associated with a pathogenic variant of a FUS gene, the composition comprising: (a) one or more antisense oligonucleotides according to the first aspect of the invention; and (b) one or more therapeutically acceptable carriers and/or diluents.

According to another aspect of the invention, there is provided a pharmaceutical composition to treat, prevent or ameliorate the effects of a disease associated with a pathogenic variant of a FUS gene, the composition comprising: (a) one or more antisense oligonucleotides of the first aspect of the invention; and (b) one or more pharmaceutically acceptable carriers and/or diluents.

According to another aspect of the invention, there is provided a method of treating, preventing or ameliorating the effects of a disease associated with a pathogenic variant of a FUS gene, the method comprising the step of administering to the subject an effective amount of the pharmaceutical composition of the invention.

In one embodiment, the pathogenic variant is a genetic alteration that increases the subject's susceptibility or predisposition to the disease. In another embodiment, the disease is selected from the group consisting of: ALS, FTD or ET. Preferably, the disease is selected from the group consisting of: ALS or FTD. Preferably, the disease is selected from the group consisting of: FUS-ALS or FUS-FTD.

According to another aspect of the invention, there is provided a method for treating, preventing or ameliorating the effects of a disease associated with a pathogenic variant in a FUS gene in patients identified by a biomarker, the method comprising the step of: (a) testing a subject for the presence of a biomarker associated with a disease associated with FUS proteinopathy where patients are likely to respond to FUS suppression; and (b) if the subject is found to express the biomarker, administering to the subject an effective amount of the pharmaceutical composition of the invention. Preferably, the disease is selected from the group consisting of: ALS or FTD. Preferably, the biomarker is a pathogenic variant of the FUS gene.

According to another aspect of the invention, there is provided a method of reducing the expression of selected FUS alleles in a subject and/or reducing the over expression of FUS caused by auto regulation in a subject, the method comprising the step of administering to the subject an effective amount of the pharmaceutical composition of the invention.

According to another aspect of the invention, there is provided a method of: (a) reducing the expression of selected FUS alleles in a subject; and/or (b) reducing the over expression of FUS caused by auto regulation in a subject the method comprising the step of administering to the subject an effective amount of a pharmaceutical composition comprising: one or more antisense oligonucleotides according to the first aspect of the invention; and one or more pharmaceutically acceptable carriers and/or diluents.

According to another aspect of the invention, there is provided an expression vector comprising one or more antisense oligonucleotides according to the first aspect of this invention.

According to another aspect of the invention, there is provided a cell comprising the antisense oligonucleotide according to the first aspect of this invention.

According to another aspect of the invention, there is provided the use of antisense oligonucleotides according to the first aspect of this invention, for the manufacture of a medicament to treat, prevent or ameliorate the effects of a disease associated with a pathogenic variant of a FUS gene.

According to another aspect of the invention, there is provided the use of antisense oligonucleotides according to the first aspect of this invention, to treat, prevent or ameliorate the effects of a disease associated with a pathogenic variant of the FUS gene. Preferably, the disease is selected from the group consisting of: ALS or FTD.

According to another aspect of the invention, there is provided a kit to treat, prevent or ameliorate the effects of a disease associated with pathogenic FUS variants in a subject, wherein the kit comprises at least an antisense oligonucleotide according to the first aspect of this invention, packaged in a suitable container, together with instructions for its use.

Further features of the present invention are more fully described in the following description of several non-limiting embodiments thereof. This description is included solely for the purposes of exemplifying the present invention. It should not be understood as a restriction on the broad summary, disclosure or description of the invention as set out above.

### BRIEF DESCRIPTION OF DRAWINGS

The following description is provided with reference to the following accompanying drawings.

FIG. 1 shows FUS expression measured via PCR and agarose gel electrophoresis with densitometry in fibroblast cell lines that are homozygous at each CV transfected with FUS CV targeted AONs (Series 1). Cells transfected at 50 nM and 12.5 nM using L3K for 24 hours. (a) AONs targeted to CV1. (b) AONs targeted to CV2. (c) AONs targeted to CV3. Error bars are Standard error (n=2).

FIG. 2 shows the percentage of the desired (non-target) allele in CV2 heterozygous fibroblasts after 24 hours incubation with FUS CV2 targeted AONs or controls at 100 nM measured with droplet digital PCR. Additional mismatch distance refers to number of bases away from the CV and mismatch type is written with base in the AON followed by the base in the target RNA. Error bars are standard error (n≥2).

FIG. 3 shows total FUS mRNA expression in fibroblast cells heterozygous for CV2 24 hours after transfection with FUS CV2 targeted AONs or controls at 100 nM. Measured via RT-PCR, agarose gel electrophoresis and densitometric analysis. Additional mismatch distance refers to number of bases away from the CV and mismatch type is written with base in the AON followed by the base in the target RNA. Error bars are standard error (n=3).

FIG. 4 shows the percentage of the desired allele and total FUS expression in fibroblasts heterozygous for CV2 3 days after transfection with CV2 targeted AONs CV2b and CV2e with 2′-Omethyl gapmer AONs with the PS modification in the wings only or throughout and with a TMO gapmer.

### DETAILED DESCRIPTION

The present invention provides a prophylactic or therapeutic method for ameliorating or slowing the further progress of symptoms of diseases associated with FUS proteinopathy (including ALS and FTD) using AON therapy. More specifically, the invention provides isolated or purified AONs targeted to a nucleic acid molecule encoding FUS pre-mRNA or mRNA wherein the AON has a nucleobase sequence that is: (a) selected from the list comprising SEQ ID NO: 1 to SEQ ID NO: 34 inclusive or variants thereof, or (b) a sequence that is complementary to at least 1 or more contiguous nucleobases in a target FUS pre-mRNA or mRNA to which SEQ ID NO: 1 to SEQ ID NO: 34 inclusive or variants thereof, also bind, and (c) wherein the AON inhibits the expression of human FUS or variants thereof.

For convenience, the following sections generally outline the various meanings of the terms used herein. Following this discussion, general aspects regarding compositions, use of medicaments and methods of the invention are discussed, followed by specific examples demonstrating the properties of various embodiments of the invention and how they can be employed.

### 1. Definitions

The meaning of certain terms and phrases used in the specification, examples, and appended claims, are provided below. If there is an apparent discrepancy between the usage of a term in the art and its definition provided herein, the definition provided within the specification shall prevail.

Those skilled in the art will appreciate that the invention described herein is susceptible to variations and modifications other than those specifically described. The invention includes all such variations and modifications. The invention also includes all of the steps, features, formulations and compounds referred to or indicated in the specification, individually or collectively and any and all combinations or any two or more of the steps or features.

Each document, reference, patent application or patent cited in this text is expressly incorporated herein in their entirety by reference, which means that it should be read and considered by the reader as part of this text. That the document, reference, patent application or patent cited in this text is not repeated in this text is merely for reasons of conciseness. None of the cited material or the information contained in that material should, however, be understood to be common general knowledge.

Manufacturer's instructions, descriptions, product specifications, and product sheets for any products mentioned herein or in any document incorporated by reference herein, are hereby incorporated herein by reference, and can be employed in the practice of the invention.

The present invention is not to be limited in scope by any of the specific embodiments described herein. These embodiments are intended for the purpose of exemplification only. Functionally equivalent products, formulations and methods are clearly within the scope of the invention as described herein.

Other than in the operating examples, or where otherwise indicated, all numbers expressing quantities of ingredients or reaction conditions used herein should be understood as modified in all instances by the term “about.” The term “about” when used in connection with percentages can mean ±1%.

The invention described herein may include one or more range of values (e.g., size, concentration etc.). A range of values will be understood to include all values within the range, including the values defining the range, and values adjacent to the range which lead to the same or substantially the same outcome as the values immediately adjacent to that value which defines the boundary to the range. For example, a person skilled in the field will understand that a 10% variation in upper or lower limits of a range can be totally appropriate and is encompassed by the invention. More particularly, the variation in upper or lower limits of a range will be 5% or as is commonly recognised in the art, whichever is greater.

In this application, the use of the singular also includes the plural unless specifically stated otherwise. In this application, the use of “or” means “and/or” unless stated otherwise. Furthermore, the use of the term “including”, as well as other forms, such as “includes” and “included”, is not limiting. Also, terms such as “element” or “component” encompass both elements and components comprising one unit and elements and components that comprise more than one subunit unless specifically stated otherwise. Also, the use of the term “portion” can include part of a moiety or the entire moiety.

Throughout this specification, unless the context requires otherwise, the word “comprise” or variations such as “comprises” or “comprising”, will be understood to imply the inclusion of a stated integer or group of integers but not the exclusion of any other integer or group of integers.

As used herein, the term “administer” refers to the placement of a composition into a subject by a method or route which results in at least partial localization of the composition at its desired site of action such that desired effect is produced. A compound or composition described herein can be administered by any appropriate route known in the art including, but not limited to, oral or parenteral routes, including intravenous, intramuscular, subcutaneous, transdermal, airway (aerosol), pulmonary, nasal, rectal, and topical (including buccal and sublingual) administration.

As used herein, the term “FUS common variant” refers to variants in the FUS gene or FUS protein. Preferably, the variation is common and occurs in the human population. More preferably, the variation occurs frequently in the human population.

Other definitions for selected terms used herein may be found within the detailed description of the invention and apply throughout. Unless otherwise defined, all other scientific and technical terms used herein have the same meaning as commonly understood to one of ordinary skill in the art to which the invention belongs.

Features of the invention will now be discussed with reference to the following non-limiting description and examples.

### 2. Embodiments

Embodiments of the present invention relate generally to improved antisense compounds, and methods or use thereof, which are specifically designed to supress the expression of FUS. Pathogenic variants in FUS have been implicated in disease associated with FUS proteinopathy including ALS and FTD.

Without being bound by theory, the present invention is based on the understanding that in patients that are heterozygous for a pathogenic FUS variant, suppressing the expression of FUS that contains a pathogenic variant may have the effect of slowing progressing of symptoms and/or improving survival of these patients. This is because FUS pathogenic variations can lead to cytoplasmic mislocalisation of FUS, splicing defects, DNA damage and to compromise FUS autoregulation. Pathogenic FUS variants are associated with a number of neurological conditions including ALS and FTD. Therefore, the suppression of the FUS alleles that contain a pathogenic variant is hypothesised to result in slowing progressing of symptoms and/or improving survival of patients suffering from disease associated with FUS pathogenic variants including ALS and FTD.

### A. Antisense Oligonucleotides

This invention provides one or more isolated or purified AONs that target a nucleic acid molecule encoding FUS pre-mRNA and mRNA, wherein the AON has a nucleobase sequence selected from the list comprising SEQ ID NO: 1 to SEQ ID NO: 34 inclusive (as set out in Tables 1 and 2, below) and wherein the AON inhibits the expression of human FUS or variants thereof. In one example, the AON is a thiophosphoramidate morpholino oligomer (TMO) chimera (gapmer): morpholino 3′-thiophosphoramidates wings and 2′-deoxynucleoside 3′-thiophosphates gap with a 2′-deoxynucleoside base at the 3′ end. In another example, the AON is a 2′ O-methoxyethyl chimera (gapmer).

Preferably, the AON is targeted to a FUS common variant selected from the group consisting of CV1 (rs741810), CV2 (rs1052352), and CV3 (rs4889537).

More generally, the invention provides isolated or purified antisense oligonucleotides that target to a nucleic acid molecule encoding FUS pre-mRNA or mRNA, wherein the AON has a nucleobase sequence that is:
a. selected from the list comprising SEQ ID NO: 1 to SEQ ID NO: 34 inclusive, or
b. a sequence that is complementary to at least 1 or more contiguous nucleobases in a target FUS pre-mRNA or mRNA to which SEQ ID NO: 1 to SEQ ID NO: 34 inclusive also bind, and
c. wherein the AON inhibits the expression of human FUS or variants thereof.

Preferably, the AON is targeted against a FUS common variant (FUS CV) as provided in Table 1.

TABLE 1
Sequences of FUS CV targeted AONs

| AON ID | SEQ ID | AON co-ordinates | AON sequence (5′ to 3′) |
|---|---|---|---|
| 1 | 1 | FUS H3A(+96+115) | GCCAUATCCTGAAGTGUCCG |
| 2 | 2 | FUS H3A(+100+119) | UCUGGCCATATCCTGAAGUG |
| 3 | 3 | FUS H3A(+103+122) | UGCUCTGGCCATATCCUGAA |
| 4 | 4 | FUS H3A(+96+115) | GCCAUAGCCTGAAGTGUCCG |
| 5 | 5 | FUS H3A(+100+119) | UCUGGCCATAGCCTGAAGUG |
| 6 | 6 | FUS H3A(+103+122) | UGCUCTGGCCATAGCCUGAA |
| 7 | 7 | FUS H4A(+88+107) | GCCAGGGTAGGAGGACUGCU |
| 8 | 8 | FUS H4A(+92+111) | CAUAGCCAGGGTAGGAGGAC |
| 9 | 9 | FUS H4A(+95+114) | GGCCATAGCCAGGGTAGGAG |
| 10 | 10 | FUS H4A(+88+107) | GCCAGGATAGGAGGACUGCU |
| 11 | 11 | FUS H4A(+92+111) | CAUAGCCAGGATAGGAGGAC |
| 12 | 12 | FUS H4A(+95+114) | GGCCATAGCCAGGATAGGAG |
| 13 | 13 | FUS 15A(+797+816) | UGACCTCAAGCCCTCUGAGU |
| 14 | 14 | FUS 15A(+801+820) | UCAAUGACCTCAAGCCCUCU |
| 15 | 15 | FUS 15A(+804+823) | AUGUCAATGACCTCAAGCCC |
| 16 | 16 | FUS 15A(+797+816) | UGACCTGAAGCCCTCUGAGU |
| 17 | 17 | FUS 15A(+801+820) | UCAAUGACCTGAAGCCCUCU |
| 18 | 18 | FUS 15A(+804+823) | AUGUCAATGACCTGAAGCCC |

The reference point (0) set at first base of the 5′ and 3′ splice sites; hence “+” refers to nucleotides binding within the exon and “−” indicates nucleotides binding within the intron.

In one example, the AON is targeted against regions of FUS CV2 as provided in Table 2.

TABLE 2
Sequences of FUS CV2 targeted AONs-Series 2

| AON | SEQ | | |
|---|---|---|---|
| ID | ID | AON co-ordinates | AON sequence (5′ to 3′) |
| 19 | 19 | FUS H4A(+92+111)A1 | CAUAGCCAGAGTAGGAGGAC |
| 20 | 20 | FUS H4A(+92+111)A1 | CAUAGCCAGTGTAGGAGGAC |
| 21 | 21 | FUS H4A(+92+111)A1 | CAUAGCCAAGGTAGGAGGAC |
| 22 | 22 | FUS H4A(+92+111)A1 | CAUAGCCATGGTAGGAGGAC |
| 23 | 23 | FUS H4A(+92+111)A1 | CAUAGCCAGGGTAAGAGGAC |
| 24 | 24 | FUS H4A(+92+111)A1 | CAUAGCCAGGGTATGAGGAC |
| 25 | 25 | FUS H4A(+92+111)A1 | CAUAGCGAGGGTAGGAGGAC |
| 26 | 26 | FUS H4A(+92+111)A1 | CAUAGCAAGGGTAGGAGGAC |
| 27 | 27 | FUS H4A(+92+111)A2 | CAUAGCCAGAATAGGAGGAC |
| 28 | 28 | FUS H4A(+92+111)A2 | CAUAGCCAGTATAGGAGGAC |
| 29 | 29 | FUS H4A(+92+111)A2 | CAUAGCCAAGATAGGAGGAC |
| 30 | 30 | FUS H4A(+92+111)A2 | CAUAGCCATGATAGGAGGAC |
| 31 | 31 | FUS H4A(+92+111)A2 | CAUAGCCAGGATAAGAGGAC |
| 32 | 32 | FUS H4A(+92+111)A2 | CAUAGCCAGGATATGAGGAC |
| 33 | 33 | FUS H4A(+92+111)A2 | CAUAGCGAGGATAGGAGGAC |
| 34 | 34 | FUS H4A(+92+111)A2 | CAUAGCAAGGATAGGAGGAC |

The reference point (0) set at first base of the 5′ and 3′ splice sites; hence “+” refers to nucleotides binding within the exon and “−” indicates nucleotides binding within the intron.

In any of the AONs of the present invention, the uracil (U) of the sequences provided herein may be replaced by a thymine (T). In any of the AONs of the present invention, the thymine (T) of the sequences provided herein may be replaced by a uracil (U).

Certain AONs of the invention are designed to complement suitable sequences within the human FUS pre-mRNA and mRNA within exons 3, 4 and 15. In a preferred embodiment, the AONs of the invention are designed to complement suitable sequences within exon 3 or 4 of human FUS pre-mRNA and/or mRNA. In a further preferred embodiment, the AONs of the invention are designed to selectively knock down expression of one allele of the pre-mRNA and/or mRNA in patients that are heterozygous for one of the FUS common variants. In a further preferred embodiment, the AONs of the invention are designed to induce RNase H mediated degradation of the FUS pre-mRNA and/or FUS mRNA. Most preferably, the AON of the invention is selected from the group consisting of SEQ ID NO: 1 to SEQ ID NO: 34.

The terms “antisense oligomer” and “antisense compound” and “antisense oligonucleotide” or “AON” are used interchangeably and refer to a linear sequence of cyclic subunits, each bearing a base-pairing moiety, linked by intersubunit linkages that allow the base-pairing moieties to hybridize to a target sequence in a nucleic acid (typically an RNA) by Watson-Crick base pairing, to form a nucleic acid:oligomer heteroduplex within the target sequence. The cyclic subunits are based on ribose or another pentose sugar or, in a preferred embodiment, a morpholino group (see description of morpholino oligomers below). The oligomer may have exact or near sequence complementarity to the target sequence; variations in sequence near the termini of an oligomer are generally preferable to variations in the interior. Also contemplated are peptide nucleic acids (PNAs), locked nucleic acids (LNAs), and 2′-O-Methyl oligonucleotides, among other antisense agents known in the art.

The term “oligonucleotide” includes polynucleotides such as deoxyribonucleic acid (DNA) or ribonucleic acid (RNA), with RNA being prepared or obtained by the transcription a DNA template. According to the invention, a nucleic acid may be present as a single-stranded or double-stranded and linear or covalently circularly closed molecule.

By “isolated” it is meant material that is substantially or essentially free from components that normally accompany it in its native state. For example, an “isolated polynucleotide” or “isolated oligonucleotide,” as used herein, may refer to a polynucleotide that has been purified or removed from the sequences that flank it in a naturally occurring state, e.g., a DNA fragment that is removed from the sequences that are adjacent to the fragment in the genome. The term “isolating” as it relates to cells refers to the purification of cells (e.g., fibroblasts, lymphoblasts) from a source subject (e.g., a subject with a polynucleotide repeat disease). In the context of mRNA or protein, “isolating” refers to the recovery of mRNA or protein from a source, e.g., cells.

An AON can be said to be “directed to” or “targeted against” a target sequence with which it hybridizes. In certain embodiments, the target sequence includes a region including the polyadenylation site and surrounding regions. The target sequence is typically a region including an AUG start codon of an mRNA, a Translation Suppressing Oligomer, or splice site of a pre-processed mRNA, a Splice Suppressing Oligomer (SSO). The target sequence for a splice site may include an mRNA sequence having its 5′ end 1 to about 25 base pairs downstream of a normal splice acceptor junction in a pre-processed mRNA. A preferred target sequence is any region of a pre-processed mRNA that includes a splice site or is contained entirely within an exon coding sequence or spans a splice acceptor or donor site. An oligomer is more generally said to be “targeted against” a biologically relevant target, such as a protein, virus, or bacteria, when it is targeted against the nucleic acid of the target in the manner described above.

As used herein, “sufficient length” or “sufficient sequence complementarity” refers to an AON that is complementary to at least 1, more typically 1-30, contiguous nucleobases in a target FUS pre-mRNA and/or FUS mRNA. In some embodiments, an antisense of sufficient length includes at least 1, 2, 3, 4, 5, 6, 7, 8, 9, 10, 11, 12, 13, 14, or 15 contiguous nucleobases in the target FUS pre-mRNA and/or FUS mRNA. In other embodiments an antisense of sufficient length includes at least 16, 17, 18, 19, 20, 21, 22, 23, 24, or 25 contiguous nucleobases in the target FUS pre-mRNA and/or FUS mRNA. Preferably, an oligonucleotide of sufficient length is from about 10 to about 50 nucleotides in length, including oligonucleotides of 10, 11, 12, 13, 14, 15, 16, 17, 18, 19, 20, 21, 22, 23, 24, 25, 26, 27, 28, 29, 30, 31, 32, 33, 34, 35, 36, 37, 38, 39 and 40 or more nucleotides. In one embodiment, an oligonucleotide of sufficient length is from 10 to about 30 nucleotides in length. In another embodiment, an oligonucleotide of sufficient length is from 15 to about 25 nucleotides in length. In yet another embodiment, an oligonucleotide of sufficient length is from 20 to 30, or 20 to 50, nucleotides in length. In yet another embodiment, an oligonucleotide of sufficient length is from 22 to 28, 25 to 28, 24 to 29 or 25 to 30 nucleotides in length.

In certain embodiments, the AON has sufficient sequence complementarity to a target RNA to block a region of a target RNA (e.g., FUS pre-mRNA and/or mRNA) in an effective manner. In some other embodiments, the target RNA is target pre-mRNA (e.g., FUS pre-mRNA and/or mRNA) and the invention induces RNase H mediated degradation of the FUS pre-mRNA and/or FUS mRNA.

As used herein, the terms “complementary” or “complementarity” are used in reference to polynucleotides {i.e., a sequence of nucleotides) related by the base-pairing rules. For example, the sequence 5′-A-G-T-3′, is complementary to the sequence “'-T-C-A-5′. Complementarity may be “partial”, in which only some of the nucleic acids' bases are matched according to the base pairing rules. Or there may be “complete” or “total” complementarity between the nucleic acids. The degree of complementarity between nucleic acid strands has significant effects on the efficiency and strength of hybridization between nucleic acid strands. As such, a “complement” sequence, as used herein refers to an oligonucleotide sequence have some complementarity to a target RNA or DNA sequence.

For the purpose of the invention, the complement of a nucleotide sequence is the nucleotide sequence which would be capable of forming a double-stranded DNA or RNA molecule with the represented nucleotide sequence, and which can be derived from the represented nucleotide sequence by replacing the nucleotides by their complementary nucleotide according to Chargaff s rules (A<>T; G<>C; A<>U) and reading in the 5′ to 3′ direction, i.e., in opposite direction of the represented nucleotide sequence. This also includes synthetic analogs of DNA/RNA (e.g., 2′ F-ANA oligos).

The term “homology” or “identity” refers to a degree of complementarity. There may be partial homology or complete sequence identity between the oligonucleotide sequence and the complement sequence of the target RNA or DNA. A partially identical sequence is an oligonucleotide that at least partially hybridises to the target RNA or DNA, leading to the formation of partial heteroduplex, and to partial or total degradation of the target RNA or DNA. A completely identical sequence is an oligonucleotide that completely hybrids to the target RNA or DNA, leading to the formation of complete heteroduplex, and to partial or total degradation of the target RNA or DNA.

In certain embodiments, AONs may be 100% complementary to the target sequence, or may include mismatches, e.g., to accommodate variants, as long as a heteroduplex formed between the oligonucleotide and target sequence is sufficiently stable to withstand the action of cellular nucleases and other modes of degradation which may occur in vivo. Hence, certain oligonucleotides may have about or at least about 70% sequence complementarity, e.g., 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or 100% sequence complementarity, between the oligonucleotide and the target sequence.

Mismatches, if present, are typically less destabilizing toward the end regions of the hybrid duplex than in the middle. The number of mismatches allowed will depend on the length of the oligonucleotide, the percentage of G:C base pairs in the duplex, and the position of the mismatch(es) in the duplex, according to well understood principles of duplex stability. Although such an AON is not necessarily 100% complementary to the target sequence, it is effective to stably and specifically bind to the target sequence, such that cleavage factor binding to the target pre-RNA is modulated.

The stability of the duplex formed between an AON and a target sequence is a function of the binding Tm and the susceptibility of the duplex to cellular enzymatic cleavage. The Tm of an oligonucleotide with respect to complementary-sequence RNA may be measured by conventional methods, such as those described by Hames et al., Nucleic Acid Hybridization, IRL Press, (1985), 107-108 or as described in Miyada C. G. and Wallace R. B., (1987), Methods Enzymol. 154, 94-107. In certain embodiments, AONs may have a binding Tm, with respect to a complementary-sequence RNA, of greater than body temperature and preferably greater than about 45° C. or 50° C. Tm's in the range 60-80° C. or greater are also included.

Additional examples of variants include AONs having about or at least about 70% sequence identity or homology, e.g., 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or 100% sequence identity or homology, over the entire length of any of SEQ ID NOS: X-Y.

In a preferred embodiment, the AONs of the invention are designed to complement suitable sequences within exon 3 and 4 of human FUS pre-mRNA and induce RNase H mediated degradation of the transcript.

Certain AONs of the invention are designed to complement suitable sequences within the human FUS pre-mRNA and/or mRNA within exons 3, 4 and 15. In a preferred embodiment, the AONs of the invention are designed to complement suitable sequences within exon 3 and 4 of human FUS pre-mRNA. In a further preferred embodiment, the AONs of the invention are designed to selectively knock down expression of one allele of the pre-mRNA and/or mRNA where the patient is heterozygous for a FUS common variant. In a further preferred embodiment, the AONs of the invention are designed to induce RNase H mediated degradation of the FUS pre-mRNA and/or FUS mRNA. Most preferably, the AON of the invention is selected from the group consisting of SEQ ID NO: 1 to SEQ ID NO: 34.

The AON is preferably selected from those provided in Table 1 or Table 2. For example, the AON used in the present invention is chosen from the list comprising SEQ ID NO: 1 to 34. Most preferably, the AON is selected from the list comprising SEQ ID NO:1 to 12. More preferably, the AON is selected from the list comprising SEQ ID NO: 2, 4, 5, 8 and 11.

### B. Methods of Use

The invention further provides a method of inhibiting the expression of FUS, the method comprising the steps of:
(a) providing one or more of the AONs as described herein and
(b) allowing the oligomer(s) to bind to a target nucleic acid site.

More specifically, the AON may be selected from those set forth in Table 1 or Table 2. The sequences are preferably selected from the group consisting of any one or more of SEQ ID Nos: SEQ ID NO: 1 to SEQ ID NO: 34, and combinations or cocktails thereof. This includes sequences which can hybridise to such sequences under stringent hybridisation conditions, sequences complementary thereto, sequences containing modified bases, modified backbones, and functional truncations or extensions thereof which possess or modulate RNA processing activity in a FUS gene transcript.

Preferably, the AON used in the present invention is chosen from the list comprising SEQ ID NO: 1 to 12. Most preferably, the AON is chosen from the list comprising SEQ ID NO: 2, 4, 5, 8 and 11.

In one preferred embodiment, the AONs used in the present method induces RNase H mediated degradation of FUS pre-mRNA and/or mRNA.

A therapeutic strategy that combines: (1) AONs designed to reduce FUS expression (SEQ ID NO: 1 to 34) will reduce the impact of pathogenic FUS variations. In one aspect, the invention seeks to provide a means for ameliorating FUS proteinopathy or disease in a subject suffering from diseases associated with pathogenic FUS variants.

### Target Sequence and Selective Hybridisation

The oligomer and the DNA, cDNA or RNA are complementary to each other when a sufficient number of corresponding positions in each molecule are occupied by nucleotides which can hydrogen bond with each other. Thus, “specifically hybridisable” and “complementary” are terms which are used to indicate a sufficient degree of complementarity or pairing such that stable and specific binding occurs between the oligomer and the DNA, cDNA or RNA target. It is understood in the art that the sequence of an AON need not be 100% complementary to that of its target sequence to be specifically hybridisable. An AON is specifically hybridisable when binding of the compound to the target DNA or RNA molecule interferes with the normal function of the target DNA or RNA product, and there is a sufficient degree of complementarity to avoid non-specific binding of the AON to non-target sequences under conditions in which specific binding is desired, i.e., under physiological conditions in the case of in vivo assays or therapeutic treatment, and in the case of in vitro assays, under conditions in which the assays are performed.

Selective hybridisation may be under low, moderate or high stringency conditions, but is preferably under high stringency. Those skilled in the art will recognise that the stringency of hybridisation will be affected by such conditions as salt concentration, temperature, or organic solvents, in addition to the base composition, length of the complementary strands and the number of nucleotide base mismatches between the hybridising nucleic acids. Stringent temperature conditions will generally include temperatures in excess of 30° C., typically in excess of 37° C., and preferably in excess of 45° C., preferably at least 50° C., and typically 60° C.-80° C. or higher. Stringent salt conditions will ordinarily be less than 1000 mM, typically less than 500 mM, and preferably less than 200 mM. However, the combination of parameters is much more important than the measure of any single parameter. An example of stringent hybridisation conditions is 65° C. and 0.1×SSC (1×SSC=0.15 M NaCl, 0.015 M sodium citrate pH 7.0). Thus, the AONs of the present invention may include oligomers that selectively hybridise to the sequences provided in Table 1 or Table 2.

At a given ionic strength and pH, the Tm is the temperature at which 50% of a target sequence hybridizes to a complementary polynucleotide. Such hybridization may occur with “near” or “substantial” complementarity of the AON to the target sequence, as well as with exact complementarity.

Typically, selective hybridisation will occur when there is at least about 55% identity over a stretch of at least about 14 nucleotides, preferably at least about 65%, more preferably at least about 75% and most preferably at least about 90%, 95%, 98% or 99% identity with the nucleotides of the antisense oligomer. The length of homology comparison, as described, may be over longer stretches and in certain embodiments will often be over a stretch of at least about nine nucleotides, usually at least about 12 nucleotides, more usually at least about 20, often at least about 21, 22, 23 or 24 nucleotides, at least about 25, 26, 27 or 28 nucleotides, at least about 29, 30, 31 or 32 nucleotides, at least about 36 or more nucleotides.

Thus, in some embodiments, the AON sequences of the invention preferably have at least 75%, more preferably at least 85%, more preferably at least 86, 87, 88, 89 or 90% homology to the sequences shown in the sequence listings herein. More preferably there is at least 91, 92, 93 94, or 95%, more preferably at least 96, 97, 98% or 99%, homology. Generally, the shorter the length of the antisense oligomer, the greater the homology required to obtain selective hybridisation. Consequently, where an AON of the invention consists of less than about 30 nucleotides, it is preferred that the percentage identity is greater than 75%, preferably greater than 85, 86, 87, 88, 89, 90, 91, 92, 93, 94, 95%, 96, 97, 98% or 99% compared with the AONs set out in the sequence listings herein. Nucleotide homology comparisons may be conducted by sequence comparison programs such as the GCG Wisconsin Bestfit program or GAP (Deveraux et al., 1984, Nucleic Acids Research 12, 387-395). In this way sequences of a similar or substantially different length to those cited herein could be compared by insertion of gaps into the alignment, such gaps being determined, for example, by the comparison algorithm used by GAP.

The AONs of the present invention may have regions of reduced homology, and regions of exact homology with the target sequence. It is not necessary for an oligomer to have exact homology for its entire length. For example, the oligomer may have continuous stretches of at least 4 or 5 bases that are identical to the target sequence, preferably continuous stretches of at least 6 or 7 bases that are identical to the target sequence, more preferably continuous stretches of at least 8 or 9 bases that are identical to the target sequence. The oligomer may have stretches of at least 10, 11, 12, 13, 14, 15, 16, 17, 18, 19, 20, 21, 22, 23, 24, 25 or 26 bases that are identical to the target sequence. The remaining stretches of oligomer sequence may be intermittently identical with the target sequence; for example, the remaining sequence may have an identical base, followed by a non-identical base, followed by an identical base. Alternatively (or as well) the oligomer sequence may have several stretches of identical sequence (for example 3, 4, 5 or 6 bases) interspersed with stretches of less than perfect homology. Such sequence mismatches will preferably have no or very little loss of cleavage modifying activity.

### Physiological Response

In an aspect, the method of the present invention induces a physiological response in a subject. Preferably, the method reduces the expression of selected FUS alleles.

The term “modulate” or “modulates” includes to “increase” or “decrease” one or more quantifiable parameters, optionally by a defined and/or statistically significant amount. The terms “increase” or “increasing,” “enhance” or “enhancing,” or “stimulate” or “stimulating” refer generally to the ability of one or AONs or compositions to produce or cause a greater physiological response (i.e., downstream effects) in a cell or a subject relative to the response caused by either no AON or a control compound.

By “enhance” or “enhancing,” or “increase” or “increasing,” or “stimulate” or “stimulating,” refers generally to the ability of one or antisense compounds or compositions to produce or cause a greater physiological response (i.e., downstream effects) in a cell or a subject, as compared to the response caused by either no antisense compound or a control compound. An “increased” or “enhanced” amount is typically a “statistically significant” amount, and may include an increase that is 1.1, 1.2, 2, 3, 4, 5, 6, 7, 8, 9, 10, 15, 20, 30, 40, 50 or more times (e.g., 500, 1000 times) (including all integers and decimal points in between and above 1), e.g., 1.5, 1.6, 1.7, 1.8, etc.) the amount produced by no antisense compound (the absence of an agent) or a control compound.

The terms “decreasing” or “decrease” refer generally to the ability of one or AONs or compositions to produce or cause a reduced physiological response (i.e., downstream effects) in a cell or a subject relative to the response caused by either no AON or a control compound. The term “reduce” or “inhibit” may relate generally to the ability of one or more antisense compounds of the invention to “decrease” a relevant physiological or cellular response, such as a symptom of a disease or condition described herein, as measured according to routine techniques in the diagnostic art. Relevant physiological or cellular responses (in vivo or in vitro) will be apparent to persons skilled in the art and may include reductions in the symptoms or pathology of a FUS related condition. A “decrease” in a response may be statistically significant as compared to the response produced by no antisense compound or a control composition, and may include a 1%, 2%, 3%, 4%, 5%, 6%, 7%, 8%, 9%, 10%, 11%, 12%, 13%, 14%, 15%, 16%, 17%, 18%, 19%, 20%, 25%, 30%, 35%, 40%, 45%, 50%, 55%, 60%, 65%, 70%, 75%, 80%, 85%, 90%, 95%, or 100% decrease, including all integers in between.

Relevant physiological or cellular responses (in vivo or in vitro) will be apparent to persons skilled in the art and may include decreases in the amount of FUS expression. An “increased” or “enhanced” amount is typically a statistically significant amount, and may include an increase that is 1.1, 1.2, 2, 3, 4, 5, 6, 7, 8, 9, 10, 15, 20, 30, 40, 50 or more times (e.g., 500, 1000 times) (including all integers and decimal points in between and above 1, e.g., 1.5, 1.6, 1.7. 1.8) the amount produced by no AON (the absence of an agent) or a control compound. The term “reduce” or “inhibit” may relate generally to the ability of one or more AONs or compositions to “decrease” a relevant physiological or cellular response, such as a symptom of a disease or condition described herein, as measured according to routine techniques in the diagnostic art. Relevant physiological or cellular responses (in vivo or in vitro) will be apparent to persons skilled in the art and may include reductions in the symptoms or pathology of a disease associated with FUS proteinopathy, such as ALS, FTLD, AD. A “decrease” in a response may be statistically significant as compared to the response produced by no AON or a control composition, and may include a 1%, 2%, 3%, 4%, 5%, 6%, 7%, 8%, 9%, 10%, 11%, 12%, 13%, 14%, 15%, 16%, 17%, 18%, 19%, 20%, 25%, 30%, 35%, 40%, 45%, 50%, 55%, 60%, 65%, 70%, 75%, 80%, 85%, 90%, 95%, or 100% decrease, including all integers in between.

### Modified AONs

In some embodiments, the AONs have the chemical composition of a naturally occurring nucleic acid molecule, i.e., the AONs do not include a modified or substituted base, sugar, or inter-subunit linkage.

In a preferred embodiment, the AONs of the present invention are non-naturally occurring nucleic acid molecules, or “oligonucleotide analogs”. For example, non-naturally occurring nucleic acids can include one or more non-natural base, sugar, and/or inter-subunit linkage, e.g., a base, sugar, and/or linkage that has been modified or substituted with respect to that found in a naturally occurring nucleic acid molecule. Exemplary modifications are described below. In some embodiments, non-naturally occurring nucleic acids include more than one type of modification, e.g., sugar and base modifications, sugar and linkage modifications, base and linkage modifications, or base, sugar, and linkage modifications. For example, in some embodiments, the AONs contain a non-natural (e.g., modified or substituted) base. In some embodiments, the AONs contain a non-natural (e.g., modified or substituted) sugar. In some embodiments, the AONs contain a non-natural (e.g., modified or substituted) inter-subunit linkage. In some embodiments, the AONs contain more than one type of modification or substitution, e.g., a non-natural base and/or a non-natural sugar, and/or a non-natural inter-subunit linkage.

Thus, included are non-naturally occurring AONs having (i) a modified backbone structure, e.g., a backbone other than the standard phosphodiester linkage found in naturally occurring oligo- and polynucleotides, and/or (ii) modified sugar moieties, e.g., morpholino moieties rather than ribose or deoxyribose moieties. Oligonucleotide analogs support bases capable of hydrogen bonding by Watson-Crick base pairing to standard polynucleotide bases, where the analog backbone presents the bases in a manner to permit such hydrogen bonding in a sequence-specific fashion between the oligonucleotide analog molecule and bases in a standard polynucleotide (e.g., single-stranded RNA or single-stranded DNA). Preferred analogs are those having a substantially uncharged, phosphorus containing backbone.

One method for producing AONs is the methylation of the 2′ hydroxyribose position and the incorporation of a phosphorothioate backbone produces molecules that superficially resemble RNA but that are much more resistant to nuclease degradation, although persons skilled in the art of the invention will be aware of other forms of suitable backbones that may be useable in the objectives of the invention.

To avoid degradation of pre-mRNA/mRNA during duplex formation with the antisense oligomers, the AONs used in the method may be adapted to minimise or prevent cleavage by endogenous RNase H. Antisense molecules that do not activate RNase H can be made in accordance with known techniques (see, e.g., U.S. Pat. No. 5,149,797). Such antisense molecules, which may be deoxyribonucleotide or ribonucleotide sequences, simply contain any structural modification which sterically hinders or prevents binding of RNase H to a duplex molecule containing the oligonucleotide as one member thereof, which structural modification does not substantially hinder or disrupt duplex formation. Because the portions of the oligonucleotide involved in duplex formation are substantially different from those portions involved in RNase H binding thereto, numerous antisense molecules that do not activate RNase H are available. This property is highly preferred, as the treatment of the RNA with the unmethylated oligomers, either intracellular or in crude extracts that contain RNase H, leads to degradation of the pre-mRNA: AON duplexes. Any form of modified AONs that is capable of by-passing or not inducing such degradation may be used in the present method. The nuclease resistance may be achieved by modifying the AONs of the invention so that it comprises partially unsaturated aliphatic hydrocarbon chain and one or more polar or charged groups including carboxylic acid groups, ester groups, and alcohol groups.

An example of AONs which when duplexed with RNA are not cleaved by cellular RNase H is 2′-O-methyl derivatives. Such 2′-O-methyl-oligoribonucleotides are stable in a cellular environment and in animal tissues, and their duplexes with RNA have higher Tm values than their ribo- or deoxyribo-counterparts. Alternatively, the nuclease resistant AONs of the invention may have at least one of the last 3′-terminus nucleotides fluoridated. Still alternatively, the nuclease resistant AONs of the invention have phosphorothioate bonds linking between at least two of the last 3′-terminus nucleotide bases, preferably having phosphorothioate bonds linking between the last four 3′-terminal nucleotide bases.

Decreased RNA cleavage may also be achieved with alternative oligonucleotide chemistry (see, e.g., U.S. Pat. No. 5,149,797). For example, the AON may be chosen from the list comprising: thiophosphoramidate morpholino oligomer (TMO) chimera, phosphoramidate or phosphorodiamidate morpholino oligomer (PMO); PMO-X; PPMO; peptide nucleic acid (PNA); a locked nucleic acid (LNA) and derivatives including alpha-L-LNA, 2′-amino LNA, 4′-methyl LNA and 4′-O-methyl LNA; ethylene bridged nucleic acids (ENA) and their derivatives; phosphorothioate oligomer; tricyclo-DNA oligomer (tcDNA); tricyclophosphorothioate oligomer; 2′ O-Methyl-modified oligomer (2′-Ome); 2′-O-methoxy ethyl (2′-MOE); 2′-fluoro, 2′-fluroarabino (FANA); unlocked nucleic acid (UNA); hexitol nucleic acid (HNA); cyclohexenyl nucleic acid (CeNA); 2′-amino (2′-NH2); 2′-O-ethyleneamine or any combination of the foregoing as mixmers or as gapmers. The key benefit of PMOs is an increased safety profile. Their neutral charge makes them less susceptible to protein interactions with reduced platelet activation and immune activation. This also reduces degradation by nucleases. They have also been used safely in DMD patients for more than 5 years.

In an aspect, the modified AON of the invention can be conjugated to a peptide. Preferably, the AON is a PTMO, i.e., a TMO oligonucleotide chemically conjugated to a peptide moiety via amide, maleimide or click chemistry (preferably using copper-free click chemistry for example via cyclooctyne linkage) and includes suitable linkers, such as cleavable or pH-sensitive linkers. The peptide moiety may be linked via either the 3′ or the 5′ terminus. Most preferably, the peptide moiety is a peptide that is capable of improving the capacity of the AON to penetrate the cell and reach the nucleus. For example, the peptide moiety can be an arginine-rich peptide, cationic peptide and/or a peptide selected from a library of peptides derived from genomes of biodiverse microorganisms (Hoffman et al., Sci Rep, 8, 1, 12538). The peptides may or may not contain non-natural amino acids and/or chemically modified amino acids.

Cell penetrating peptides have been added to phosphorodiamidate morpholino oligomers to enhance cellular uptake and nuclear localization. Different cell penetrating peptides have been shown to influence efficiency of uptake and target tissue specificity, as shown in Jearawiriyapaisarn et al. (2008), Mol. Ther., 16(9), 1624-1629. The terms “cell penetrating peptide” and “CPP” are used interchangeably and refer to cationic cell penetrating peptides, also called transport peptides, carrier peptides, or peptide transduction domains. The peptides, as shown herein, have the capability of inducing cell penetration within 100% of cells of a given cell culture population and allow macromolecular translocation within multiple tissues in vivo upon systemic administration. The peptides are also capable of enhancing cellular uptake after localized delivery to a tissue or organ.

To further improve the delivery efficacy, the abovementioned modified nucleotides are often conjugated with fatty acids/lipid/cholesterol/amino acids/carbohydrates/polysaccharides/nanoparticles etc. to the sugar or nucleobase moieties. These conjugated nucleotide derivatives can also be used to construct AONs to induce exon skipping. Antisense oligomer-induced alternative splicing of the human FUS gene transcripts can use oligoribonucleotides, PNAs, 2′-Ome or 2′-MOE modified bases on a phosphorothioate backbone. Although 2′-OMe AONs are used for oligo design, due to their efficient uptake in vitro when delivered as cationic lipoplexes, these compounds are susceptible to nuclease degradation and are not considered ideal for in vivo or clinical applications. When alternative chemistries are used to generate the AONs of the present invention, the uracil (U) of the sequences provided herein may be replaced by a thymine (T).

For example, such antisense molecules may be oligonucleotides wherein at least one, or all, of the inter-nucleotide bridging phosphate residues are modified phosphates, such as methyl phosphonates, methyl phosphorothioates, phosphoromorpholidates, phosphoropiperazidates and phosphor amidates. For example, every other one of the internucleotide bridging phosphate residues may be modified as described. In another non-limiting example, such antisense molecules are molecules wherein at least one, or all, of the nucleotides contain a 2′ lower alkyl moiety (e.g., Ci-C4, linear or branched, saturated or unsaturated alkyl, such as methyl, ethyl, ethenyl, propyl, 1-propenyl, 2-propenyl, and isopropyl). For example, every other one of the nucleotides may be modified as described.

Specific examples of AONs useful in this invention include oligonucleotides containing modified backbones or non-natural intersubunit linkages.

Oligonucleotides having modified backbones include those that retain a phosphorus atom in the backbone and those that do not have a phosphorus atom in the backbone. Modified oligonucleotides that do not have a phosphorus atom in their inter-nucleoside backbone can also be considered to be oligonucleosides.

In other antisense molecules, both the sugar and the inter-nucleoside linkage, i.e., the backbone, of the nucleotide units are replaced with novel groups. The base units are maintained for hybridization with an appropriate nucleic acid target compound. One such oligomeric compound, an oligonucleotide mimetic that has been shown to have excellent hybridization properties, is referred to as a peptide nucleic acid (PNA). In PNA compounds, the sugar-backbone of an oligonucleotide is replaced with an amide containing backbone, in particular an aminoethylglycine backbone. The nucleo-bases are retained and are bound directly or indirectly to aza nitrogen atoms of the amide portion of the backbone.

Modified oligonucleotides may also contain one or more substituted sugar moieties. Oligonucleotides may also include nucleobase (often referred to in the art simply as “base”) modifications or substitutions. Oligonucleotides containing a modified or substituted base include oligonucleotides in which one or more purine or pyrimidine bases most commonly found in nucleic acids are replaced with less common or non-natural bases.

Purine bases comprise a pyrimidine ring fused to an imidazole ring; adenine and guanine are the two purine nucleobases most commonly found in nucleic acids. These may be substituted with other naturally occurring purines, including but not limited to N₆-methyladenine, N₂-methylguanine, hypoxanthine, and 7-methylguanine.

Pyrimidine bases comprise a six-membered pyrimidine ring; cytosine, uracil, and thymine are the pyrimidine bases most commonly found in nucleic acids. These may be substituted with other naturally occurring pyrimidines, including but not limited to 5-methylcytosine, 5-hydroxymethylcytosine, pseudouracil, and 4-thiouracil. In one embodiment, the oligonucleotides described herein contain thymine bases in place of uracil.

Other modified or substituted bases include, but are not limited to, 2,6-diaminopurine, orotic acid, agmatidine, lysidine, 2-thiopyrimidine (e.g. 2-thiouracil, 2-thiothymine), G-clamp and its derivatives, 5-substituted pyrimidine (e.g. 5-halouracil, 5-propynyluracil, 5-propynylcytosine, 5 aminomethyluracil, 5-hydroxymethyluracil, 5-aminomethylcytosine, 5-hydroxymethylcytosine, Super T), 7-deazaguanine, 7-deazaadenine, 7-aza-2,6-diaminopurine, 8-aza-7-deazaguanine, 8-aza-7-deazaadenine, 8-aza-7-deaza-2,6-diaminopurine, Super G, Super A, and N₄-ethylcytosine, or derivatives thereof; N₂-cyclopentylguanine (cPent-G), N₂-cyclopentyl-2-aminopurine (cPent-AP), and N₂-propyl-2-aminopurine (Pr-AP), pseudouracil or derivatives thereof; and degenerate or universal bases, like 2,6-difluorotoluene or absent bases like abasic sites (e.g. 1-deoxyribose, 1,2-dideoxyribose, 1-deoxy-2-O-methylribose; or pyrrolidine derivatives in which the ring oxygen has been replaced with nitrogen (azaribose)). Examples of derivatives of Super A, Super G and Super T can be found in U.S. Pat. No. 6,683, 173 (Epoch Biosciences). cPent-G, cPent-AP and Pr-AP were shown to reduce immunostimulatory effects when incorporated in siRNA (Peacock H. et al. J. Am. Chem. Soc. 2011, 133, 9200). Pseudouracil is a naturally occurring isomerized version of uracil, with a C-glycoside rather than the regular N-glycoside as in uridine. Pseudouridine-containing synthetic mRNA may have an improved safety profile compared to uridine-containing mPvNA (see WO 2009127230).

Certain modified or substituted nucleo-bases are particularly useful for increasing the binding affinity of the AONs of the invention. These include 5-substituted pyrimidines, 6-azapyrimidines and N-2, N-6 and 0-6 substituted purines, including 2-aminopropyladenine, 5-propynyluracil and 5-propynylcytosine. 5-methylcytosine substitutions have been shown to increase nucleic acid duplex stability by 0.6-1.2° C. and are presently preferred base substitutions, even more particularly when combined with 2′-O-methoxyethyl sugar modifications.

In some embodiments, modified or substituted nucleo-bases are useful for facilitating purification of AONs. For example, in certain embodiments, AONs may contain three or more (e.g., 3, 4, 5, 6 or more) consecutive guanine bases. In certain AONs, a string of three or more consecutive guanine bases can result in aggregation of the oligonucleotides, complicating purification. In such AONs, one or more of the consecutive guanines can be substituted with inosine. The substitution of inosine for one or more guanines in a string of three or more consecutive guanine bases can reduce aggregation of the AON, thereby facilitating purification.

In one embodiment, another modification of the AONs involves chemically linking to the oligonucleotide one or more moieties or conjugates that enhance the activity, cellular distribution or cellular uptake of the oligonucleotide. Such moieties include but are not limited to lipid moieties such as a cholesterol moiety, cholic acid, a thioether, e.g., hexyl-5-tritylthiol, a thiocholesterol, an aliphatic chain, e.g., dodecandiol or undecyl residues, a phospholipid, e.g., di-hexadecyl-rac-glycerol or triethylammonium 1,2-di-O-hexadecyl-rac-27lycerol-3-H-phosphonate, a polyamine or a polyethylene glycol chain, or adamantane acetic acid, a palmityl moiety, or an octadecylamine or hexylamino-carbonyl-oxycholesterol moiety.

It is not necessary for all positions in a given compound to be uniformly modified, and in fact more than one of the aforementioned modifications may be incorporated in a single compound or even at a single nucleoside within an oligonucleotide. The present invention also includes AONs that are chimeric compounds. “Chimeric” antisense compounds or “chimeras,” in the context of this invention, are antisense molecules, particularly oligonucleotides, which contain two or more chemically distinct regions, each made up of at least one monomer unit, i.e., a nucleotide in the case of an oligonucleotide compound. These oligonucleotides typically contain at least one region wherein the oligonucleotide is modified so as to confer upon the increased resistance to nuclease degradation, increased cellular uptake, and an additional region for increased binding affinity for the target nucleic acid.

The antisense molecules used in accordance with this invention may be conveniently and routinely made through the well-known technique of solid phase synthesis. Equipment for such synthesis is sold by several vendors including, for example, Applied Biosystems (Foster City, Calif.). One method for synthesising oligonucleotides on a modified solid support is described in U.S. Pat. No. 4,458,066.

In another non-limiting example, such AONs are molecules wherein at least one, or all, of the nucleotides contain a 2′ lower alkyl moiety (such as, for example, C₁-C₄, linear or branched, saturated or unsaturated alkyl, such as methyl, ethyl, ethenyl, propyl, 1-propenyl, 2-propenyl, and isopropyl). For example, every other one of the nucleotides may be modified as described.

While the AONs described above are a preferred form of the AONs of the present invention, the present invention includes other oligomeric antisense molecules, including but not limited to oligomer mimetics such as are described below.

Modified oligomers may also contain one or more substituted sugar moieties. Oligomers may also include nucleobase (often referred to in the art simply as “base”) modifications or substitutions. Certain nucleobases are particularly useful for increasing the binding affinity of the oligomeric compounds of the invention. These include 5-substituted pyrimidines, 6-azapyrimidines, and N-2, N-6 and O-6 substituted purines, including 2-aminopropyladenine, 5-propynyluracil and 5-propynylcytosine. 5-methylcytosine substitutions have been shown to increase nucleic acid duplex stability by 0.6-1.2° C., even more particularly when combined with 2′-O-methoxyethyl sugar modifications. In one embodiment, at least one pyrimidine base of the oligonucleotide comprises a 5-substituted pyrimidine base, wherein the pyrimidine base is selected from the group consisting of cytosine, thymine and uracil. In one embodiment, the 5-substituted pyrimidine base is 5-methylcytosine. In another embodiment, at least one purine base of the oligonucleotide comprises an N-2, N-6 substituted purine base. In one embodiment, the N-2, N-6 substituted purine base is 2, 6-diaminopurine.

In one embodiment, the AON includes one or more 5-methylcytosine substitutions alone or in combination with another modification, such as 2′-O-methoxyethyl sugar modifications. In yet another embodiment, the AON includes one or more 2, 6-diaminopurine substitutions alone or in combination with another modification.

In some embodiments, the AON is chemically linked to one or more moieties, such as a polyethylene glycol moiety, or conjugates, such as an arginine-rich cell penetrating peptide that enhance the activity, cellular distribution, or cellular uptake of the AON. In one exemplary embodiment, the arginine-rich polypeptide is covalently coupled at its N-terminal or C-terminal residue to the 3′ or 5′ end of the antisense compound. Also, in an exemplary embodiment, the antisense compound is composed of morpholino subunits and phosphorus-containing inter-subunit linkages joining a morpholino nitrogen of one subunit to a 5′ exocyclic carbon of an adjacent subunit.

In another aspect, the invention provides expression vectors that incorporate the AONs described above, e.g., the AONs of SEQ ID NOs: 1-34. In some embodiments, the expression vector is a modified retrovirus or non-retroviral vector, such as an adeno-associated viral vector.

### Assays for Measuring Activity of AONs

The activity of AONs and variants thereof can be assayed according to routine techniques in the art. For example, isoform forms and expression levels of surveyed RNAs and proteins may be assessed by any of a wide variety of well-known methods for detecting isoforms and/or expression of a transcribed nucleic acid or protein. Non-limiting examples of such methods include RT-PCR of isoforms of RNA followed by size separation of PCR products, nucleic acid hybridization methods e.g., Northern blots and/or use of nucleic acid arrays; fluorescent in situ hybridization to detect RNA transcripts inside cells; nucleic acid amplification methods; immunological methods for detection of proteins; protein purification methods; and protein function or activity assays.

RNA expression levels can be assessed by preparing RNA/cDNA (i.e., a transcribed polynucleotide) from a cell, tissue or organism, and by hybridizing the RNA/cDNA with a reference polynucleotide, which is a complement of the assayed nucleic acid, or a fragment thereof. cDNA can, optionally, be amplified using any of a variety of polymerase chain reaction or in vitro transcription methods prior to hybridization with the complementary polynucleotide; preferably, it is not amplified. Expression of one or more transcripts can also be detected using quantitative PCR to assess the level of expression of the transcript(s).

### Methods of Manufacturing AONs

The AONs used in accordance with this invention may be conveniently made through the well-known technique of solid phase synthesis. Equipment for such synthesis is sold by several vendors including, for example, Applied Biosystems (Foster City, Calif.). One method for synthesising oligomers on a modified solid support is described in U.S. Pat. No. 4,458,066.

Any other means for such synthesis known in the art may additionally or alternatively be employed. It is well known to use similar techniques to prepare oligomers such as the phosphorothioates and alkylated derivatives. In one such automated embodiment, diethyl-phosphoramidites are used as starting materials and may be synthesized as described by Beaucage, et al., (1981) Tetrahedron Letters, 22:1859-1862.

The AONs of the invention are synthesised in vitro and do not include antisense compositions of biological origin, or genetic vector constructs designed to direct the in vivo synthesis of antisense oligomers. The molecules of the invention may also be mixed, encapsulated, conjugated or otherwise associated with other molecules, molecule structures or mixtures of compounds, as for example, liposomes, receptor targeted molecules, oral, rectal, topical or other formulations, for assisting in uptake, distribution and/or absorption.

### Vectors

Also included are vector delivery systems that are capable of expressing the oligomeric, FUS-targeting sequences of the present invention, such as vectors that express a polynucleotide sequence comprising any one or more of SEQ ID NO: 1 to 34, as described herein.

By “vector” or “nucleic acid construct” is meant a polynucleotide molecule, preferably a DNA molecule derived, for example, from a plasmid, bacteriophage, yeast or virus, into which a polynucleotide can be inserted or cloned. A vector preferably contains one or more unique restriction sites and can be capable of autonomous replication in a defined host cell including a target cell or tissue or a progenitor cell or tissue thereof or able to be integrated with the genome of the defined host such that the cloned sequence is reproducible.

Accordingly, the vector can be an autonomously replicating vector, i.e., a vector that exists as an extra-chromosomal entity, the replication of which is independent of chromosomal replication, e.g., a linear or closed circular plasmid, an extra-chromosomal element, a mini-chromosome, or an artificial chromosome. The vector can contain any means for assuring self-replication. Alternatively, the vector can be one which, when introduced into the host cell, is integrated into the genome and replicated together with the chromosome(s) into which it has been integrated.

### C. Method of Treatment

The AONs of the present invention also can be used as a prophylactic or therapeutic, which may be utilised for the purpose of treatment of a disease. Accordingly, in one embodiment the present invention provides AONs that bind to a selected target in the FUS pre-mRNA and mRNA to reduce expression of FUS as described herein, in a therapeutically effective amount, admixed with a pharmaceutically acceptable carrier, diluent, or excipient.

Pathogenic FUS variants can lead to FUS cytoplasmic mislocalisation, splicing defects, DNA damage and altered cellular stress responses. Without being bound by theory, decreasing the expression of FUS mRNA that contains pathogenic variants through the administration of the AONs of this invention, may result in a reduction in FUS cytoplasmic aggregation and other FUS related pathology.

An “effective amount” or “therapeutically effective amount” refers to an amount of therapeutic compound, such as an antisense oligomer, administered to a mammalian subject, either as a single dose or as part of a series of doses, which is effective to produce a desired therapeutic effect.

The invention therefore provides a pharmaceutical, prophylactic, or therapeutic composition to treat, prevent or ameliorate the effects of a disease associated with FUS pathogenic variants, the composition comprising:
a) one or more AONs as described herein, and
b) one or more pharmaceutically acceptable carriers and/or diluents.

Preferably, the disease associated with FUS pathogenic variants is ALS or FTD.

There is also provided a method for treating, preventing or ameliorating the effects of a disease associated with FUS proteinopathy, the method comprising the step of: administering to the subject an effective amount of one or more AONs or pharmaceutical composition comprising one or more AONs as described herein.

There is also provided a method for treating, preventing or ameliorating the effects of FUS-ALS or FUS-FTD, the method comprising the step of: administering to the subject an effective amount of one or more AONs or pharmaceutical composition comprising one or more AONs as described herein.

The methods of the invention can be administered in combination with additional treatments for treating, preventing, or slowing the progress of diseases associated with FUS proteinopathy and their symptoms. Additional treatments can include AONs directed to other targets associated with diseases associated with FUS proteinopathy. For example, the additional treatments can include AONs directed to SOD1.

In a further aspect, genetic or other biomarkers can be used to identify patients most likely to respond well to FUS suppression via the AONs of the invention. Genetic structural variations associated with ALS disease risk have been identified within ALS genes and surrounding gene regions. These variations can be used as genetic biomarkers to identify patients likely to respond to the methods of this invention. Non-genetic biomarkers can also be used to identify patients likely to respond to the methods of this invention.

The invention provides a method for treating, preventing or ameliorating the effects of ALS, FTD, in subjects identified by a biomarker, the method comprising the step of:
a) testing a subject for the presence of a biomarker associated with ALS patients likely to respond to FUS suppression; and
b) if the subject is found to express the biomarker, administering to the subject an effective amount of one or more AONs or pharmaceutical composition comprising one or more AONs as described herein.

There is also provided herein the use of purified and isolated AONs as described herein, to treat, prevent or ameliorate the effects of a disease associated with FUS pathogenic variants.

There is also provided herein the use of purified and isolated AONs as described herein, to treat, prevent or ameliorate the effects of ALS, FTD.

Preferably, the AON used in the present invention is chosen from the list of AONs provided in Tables 1 or 2 or more preferably is selected from SEQ ID NO:1 to 12.

The invention also provides a method of treatment that comprises the combination of: (1) AONs designed to reduce expression of FUS alleles that contain pathogenic variations (SEQ ID NO: 1 to 34) to reduce the impact of disease associated with FUS pathogenic variants. In one embodiment, the invention seeks to provide a means for ameliorating FUS proteinopathy in subjects suffering from diseases associated with FUS-ALS or FUS-FTD.

The composition may comprise about 1 nM to 1000 μM of each of the desired antisense oligomer(s) of the invention. Preferably, the composition may comprise about 1 μM to 500 μM, 10 μM to 500 μM, 50 μM to 750 μM, 10 μM to 500 μM, 1 μM to 100 μM, 1 μM to 50 μM, preferably between 25 μM and 100 μM of each of the antisense oligomer(s) of the invention. The composition may also preferably comprise about 1 nM to 500 nM, 10 nM to 500 nM, 50 nM to 750 nM, 10 nM to 500 nM, 1 nM to 100 nM, 1 nM to 50 nM, most preferably between 50 nM and 100 nM of each of the antisense oligomer(s) of the invention.

The composition may comprise about 1 nM, 2 nM, 3 nM, 4 nM, 5 nM, 6 nM, 7 nM, 8 nM, 9 nM, 10 nM, 20 nM, 50 nM, 75 nM, 100 nM, 150 nM, 200 nM, 250 nM, 300 nM, 350 nM, 400 nM, 450 nM, 500 nM, 550 nM, 600 nM, 650 nM, 700 nM, 750 nM, 800 nM, 850 nM, 900 nM, 950 nM or 1000 nM of each of the desired antisense oligomer(s) of the invention.

The present invention further provides one or more AONs adapted to aid in the prophylactic or therapeutic treatment, prevention or amelioration of symptoms of a disease or pathology associated with FUS pathogenic variations in a form suitable for delivery to a subject.

The phrase “pharmaceutically acceptable” refers to molecular entities and compositions that are physiologically tolerable and do not typically produce an allergic or similarly untoward reaction, such as gastric upset and the like, when administered to a subject. The term “carrier” refers to a diluent, adjuvant, excipient, or vehicle with which the compound is administered. Such pharmaceutical carriers can be sterile liquids, such as water and oils, including those of petroleum, animal, vegetable or synthetic origin, such as peanut oil, soybean oil, mineral oil, sesame oil and the like. Water or saline solutions and aqueous dextrose and glycerol solutions are preferably employed as carriers, particularly for injectable solutions. Suitable pharmaceutical carriers are described in Martin, Remington's Pharmaceutical Sciences, 18th Ed., Mack Publishing Co., Easton, PA, (1990).

The pharmaceutical composition comprising the one or more AONs can be administered to the subject in a range of treatment regimens. For example, the pharmaceutical composition can be administered hourly, three times daily, twice daily, once daily, once every two days, once every three days, once weekly, once every two weeks, once monthly, once every two months, once every six months, and once yearly. The appropriate regimen can be determined by the person skilled in the art based on the nature of the condition to be treated.

### D. Manufacture of a Medicament

In one embodiment, the present invention provides the use of AONs that bind to a selected target in the FUS RNA for the manufacture of a medicament to treat, prevent or ameliorate the effects of a disease associated with FUS pathogenic variants. There is therefore provided the use of one or more AONs described herein for the manufacture of a medicament to treat, prevent or ameliorate the effects of a disease associated with FUS pathogenic variants. Preferably the disease is ALS or FTD.

The invention provides the use of purified and isolated antisense oligonucleotides according as described herein, for the manufacture of a medicament to treat, prevent or ameliorate the effects of a disease associated with a disease associated with FUS pathogenic variants.

The invention also provides the use of purified and isolated antisense oligonucleotides according as described herein, for the manufacture of a medicament to treat, prevent or ameliorate the effects of ALS or FTD.

There is also provided the use of one or more AONs described herein for the manufacture of a medicament to treat, prevent or ameliorate the effects of a disease associated with FUS pathogenic variants in subjects expressing a biomarker associated with patients likely to respond to FUS suppression.

Preferably, the AON used for the manufacture of a medicament is chosen from the list of AONs provided in Tables 1 or 2 or more preferably is selected from SEQ ID NO: 1 to 12. Preferably, the AON is SEQ ID NO: 2, 4, 5, 8 and 11.

### E. Pharmaceutical Compositions

In a form of the invention there are provided pharmaceutical compositions comprising therapeutically effective amounts of one or more AONs of the invention together with pharmaceutically acceptable diluents, preservatives, solubilizers, emulsifiers, adjuvants, and/or carriers. Such compositions include diluents of various buffer content (e.g., Tris-HCl, acetate, phosphate), pH and ionic strength and additives such as detergents and solubilizing agents (e.g., Tween 80, Polysorbate 80), antioxidants (e.g., ascorbic acid, sodium metabisulfite), preservatives (e.g., Thimersol, benzyl alcohol) and bulking substances (e.g., lactose, mannitol). The material may be incorporated into particulate preparations of polymeric compounds such as polylactic acid, polyglycolic acid, etc. or into liposomes. Hyalluronic acid may also be used. Such compositions may influence the physical state, stability, rate of in vivo release, and rate of in vivo clearance of the present proteins and derivatives. See, for example, Martin, Remington's Pharmaceutical Sciences, 18th Ed. (1990, Mack Publishing Co., Easton, PA 18042) pages 1435-1712 that are herein incorporated by reference. The compositions may be prepared in liquid form, or may be in dried powder, such as a lyophilised form.

It will be appreciated that pharmaceutical compositions provided according to the present invention may be administered by any means known in the art. Preferably, the pharmaceutical compositions for administration are administered by injection, orally, topically or by the pulmonary or nasal route. The AONs are more preferably delivered by intravenous, intra-arterial, intraperitoneal, intramuscular or subcutaneous routes of administration. The appropriate route may be determined by one of skill in the art, as appropriate to the condition of the subject under treatment. Vascular or extravascular circulation, the blood or lymph system, and the cerebrospinal fluid are some non-limiting sites where the AON may be introduced. Direct CNS delivery may be employed, for instance, intracerebro-ventricular or intrathecal administration may be used as routes of administration.

Formulations for topical administration include those in which the oligomers of the disclosure are in admixture with a topical delivery agent such as lipids, liposomes, fatty acids, fatty acid esters, steroids, chelating agents and surfactants. Lipids and liposomes include neutral (e.g., dioleoylphosphatidyl DOPE ethanolamine, dimyristoylphosphatidyl choline DMPC, distearolyphosphatidyl choline) negative (e.g., dimyristoylphosphatidyl glycerol DMPG) and cationic (e.g., dioleoyltetramethylaminopropyl DOTAP and dioleoylphosphatidyl ethanolamine DOTMA). For topical or other administration, oligomers of the disclosure may be encapsulated within liposomes or may form complexes thereto, in particular to cationic liposomes. Alternatively, oligomers may be complexed to lipids, in particular to cationic lipids. Fatty acids and esters, pharmaceutically acceptable salts thereof, and their uses are further described in U.S. Pat. No. 6,287,860 and/or U.S. patent application Ser. No. 09/315,298 filed on May 20, 1999.

In certain embodiments, the AONs of the disclosure can be delivered by transdermal methods (e.g., via incorporation of the AONs into, e.g., emulsions, with such AONs optionally packaged into liposomes). Such transdermal and emulsion/liposome-mediated methods of delivery are described for delivery of AONs in the art, e.g., in U.S. Pat. No. 6,965,025.

The AONs described herein may also be delivered via an implantable device. Design of such a device is an art-recognized process, with, e.g., synthetic implant design described in, e.g., U.S. Pat. No. 6,969,400.

Compositions and formulations for oral administration include powders or granules, microparticulates, nanoparticulates, suspensions or solutions in water or non-aqueous media, capsules, gel capsules, sachets, tablets or minitablets. Thickeners, flavoring agents, diluents, emulsifiers, dispersing aids or binders may be desirable. Oral formulations are those in which oligomers of the disclosure are administered in conjunction with one or more penetration enhancers surfactants and chelators. Surfactants include fatty acids and/or esters or salts thereof, bile acids and/or salts thereof. Bile acids/salts and fatty acids and their uses are further described in U.S. Pat. No. 6,287,860. In some embodiments, the present disclosure provides combinations of penetration enhancers, for example, fatty acids/salts in combination with bile acids/salts. An exemplary combination is the sodium salt of lauric acid, capric acid and UDCA. Further penetration enhancers include polyoxyethylene-9-lauryl ether, polyoxyethylene-20-cetyl ether. Oligomers of the disclosure may be delivered orally, in granular form including sprayed dried particles, or complexed to form micro or nanoparticles. Oligomer complexing agents and their uses are further described in U.S. Pat. No. 6,287,860. Oral formulations for oligomers and their preparation are described in detail in U.S. Pat. No. 6,887,906, Ser. No. 09/315,298 filed May 20, 1999 and/or US20030027780.

Compositions and formulations for parenteral, intrathecal or intraventricular administration may include sterile aqueous solutions which may also contain buffers, diluents and other suitable additives such as, but not limited to, penetration enhancers, carrier compounds and other pharmaceutically acceptable carriers or excipients.

The delivery of a therapeutically useful amount of AONs may be achieved by methods previously published. For example, intracellular delivery of the AON may be via a composition comprising an admixture of the AON and an effective amount of a block copolymer. An example of this method is described in US patent application US20040248833. Other methods of delivery of AONs to the nucleus are described in Mann C J et al. (2001) Proc, Natl. Acad. Science, 98(1) 42-47, and in Gebski et al. (2003) Human Molecular Genetics, 12(15): 1801-1811. A method for introducing a nucleic acid molecule into a cell by way of an expression vector either as naked DNA or complexed to lipid carriers, is described in U.S. Pat. No. 6,806,084.

In certain embodiments, the AONs of the invention and therapeutic compositions comprising the same can be delivered by transdermal methods (e.g., via incorporation of the AONs into, e.g., emulsions, with such AONs optionally packaged into liposomes). Such transdermal and emulsion/liposome-mediated methods of delivery are described for delivery of AONs in the art, e.g., in U.S. Pat. No. 6,965,025.

It may be desirable to deliver the AON in a colloidal dispersion system. Colloidal dispersion systems include macromolecule complexes, nanocapsules, microspheres, beads, and lipid-based systems including oil-in-water emulsions, micelles, mixed micelles, and liposomes or liposome formulations. These colloidal dispersion systems can be used in the manufacture of therapeutic pharmaceutical compositions.

Liposomes are artificial membrane vesicles, which are useful as delivery vehicles in vitro and in vivo. These formulations may have net cationic, anionic, or neutral charge characteristics and have useful characteristics for in vitro, in vivo and ex vivo delivery methods. It has been shown that large unilamellar vesicles can encapsulate a substantial percentage of an aqueous buffer containing large macromolecules. RNA and DNA can be encapsulated within the aqueous interior and be delivered to cells in a biologically active form (Fraley, et al., 1981, Trends Biochem. Sci., 6, 77).

In order for a liposome to be an efficient gene transfer vehicle, the following characteristics should be present: (1) encapsulation of the AON of interest at high efficiency while not compromising their biological activity; (2) preferential and substantial binding to a target cell in comparison to non-target cells; (3) delivery of the aqueous contents of the vesicle to the target cell cytoplasm at high efficiency; and (4) accurate and effective expression of genetic information (Mannino, et al., 1988 Biotechniques, 6, 682). The composition of the liposome is usually a combination of phospholipids, particularly high phase-transition-temperature phospholipids, usually in combination with steroids, especially cholesterol. Other phospholipids or other lipids may also be used. The physical characteristics of liposomes depend on pH, ionic strength, and the presence of divalent cations. Cationic liposomes are positively charged liposomes which are believed to interact with negatively charged DNA molecules to form a stable complex. Liposomes that are pH-sensitive or negatively charged are believed to entrap DNA rather than complex with it. Both cationic and noncationic liposomes have been used to deliver DNA to cells.

Liposomes also include “sterically stabilized” liposomes, a term which, as used herein, refers to liposomes comprising one or more specialized lipids that, when incorporated into liposomes, result in enhanced circulation lifetimes relative to liposomes lacking such specialized lipids. Examples of sterically stabilized liposomes are those in which part of the vesicle-forming lipid portion of the liposome comprises one or more glycolipids or is derivatized with one or more hydrophilic polymers, such as a polyethylene glycol (PEG) moiety. Liposomes and their uses are further described in U.S. Pat. No. 6,287,860.

AONs can be introduced into cells using art-recognized techniques (e.g., transfection, electroporation, fusion, liposomes, colloidal polymeric particles and viral and non-viral vectors as well as other means known in the art). The method of delivery selected will depend at least on the cells to be treated and the location of the cells and will be apparent to the skilled artisan. For instance, localization can be achieved by liposomes with specific markers on the surface to direct the liposome, direct injection into tissue containing target cells, specific receptor-mediated uptake, or the like.

As known in the art, AONs may be delivered using, for example, methods involving liposome-mediated uptake, lipid conjugates, polylysine-mediated uptake, nanoparticle-mediated uptake, and receptor-mediated endocytosis, as well as additional non-endocytic modes of delivery, such as microinjection, permeabilization (e.g., streptolysin-O permeabilization, anionic peptide permeabilization), electroporation, and various non-invasive non-endocytic methods of delivery that are known in the art (refer to Dokka and Rojanasakul, Advanced Drug Delivery Reviews 44, 35-49, incorporated by reference in its entirety).

The AON may also be combined with other pharmaceutically acceptable carriers or diluents to produce a pharmaceutical composition. Suitable carriers and diluents include isotonic saline solutions, for example phosphate-buffered saline. The composition may be formulated for parenteral, intramuscular, intravenous, subcutaneous, intraocular, oral, or transdermal administration.

The routes of administration described are intended only as a guide since a skilled practitioner will be able to readily determine the optimum route of administration and any dosage for any particular animal and condition.

Multiple approaches for introducing functional new genetic material into cells, both in vitro and in vivo have been attempted (Friedmann (1989) Science, 244, 1275-1280). These approaches include integration of the gene to be expressed into modified retroviruses (Friedmann (1989) supra; Rosenberg (1991) Cancer Research 51(18), suppl.: 5074S-5079S); integration into non-retrovirus vectors (Rosenfeld, et al. (1992) Cell, 68, 143-155; Rosenfeld, et al. (1991) Science, 252, 431-434); or delivery of a transgene linked to a heterologous promoter-enhancer element via liposomes (Friedmann (1989), supra; Brigham, et al. (1989) Am. J. Med. Sci., 298, 278-281; Nabel, et al. (1990) Science, 249, 1285-1288; Hazinski, et al. (1991) Am. J. Resp. Cell Molec. Biol., 4:206-209; and Wang and Huang (1987) Proc. Natl. Acad. Sci. (USA), 84, 7851-7855); coupled to ligand-specific, cation-based transport systems (Wu and Wu (1988) J. Biol. Chem., 263, 14621-14624) or the use of naked DNA, expression vectors (Nabel et al. (1990), supra); Wolff et al. (1990) Science, 247, 1465-1468). Direct injection of transgenes into tissue produces only localized expression (Rosenfeld (1992) supra); Rosenfeld et al. (1991) supra; Brigham et al. (1989) supra; Nabel (1990) supra; and Hazinski et al. (1991) supra). The Brigham et al. group ((1989) Am. J. Med. Sci. 298, 278-281 and Clinical Research (1991) 39 (abstract)) have reported in vivo transfection only of lungs of mice following either intravenous or intratracheal administration of a DNA liposome complex. An example of a review article of human gene therapy procedures is: Anderson, (1992) Science 256, 808-813; Barteau et al. (2008), Curr Gene Ther., 8(5), 313-23; Mueller et al. (2008). Clin Rev Allergy Immunol., 35(3), 164-78; Li et al. (2006) Gene Ther., 13(18), 1313-9; Simoes et al. (2005) Expert Opin Drug Deliv., 2(2), 237-54.

The AONs of the invention encompass any pharmaceutically acceptable salts, esters, or salts of such esters, or any other compound which, upon administration to an animal including a human, is capable of providing (directly or indirectly) the biologically active metabolite or residue thereof. Accordingly, as an example, the disclosure is also drawn to prodrugs and pharmaceutically acceptable salts of the compounds of the invention, pharmaceutically acceptable salts of such pro-drugs, and other bioequivalents.

The term “pharmaceutically acceptable salts” refers to physiologically and pharmaceutically acceptable salts of the compounds of the invention: i.e., salts that retain the desired biological activity of the parent compound and do not impart undesired toxicological effects thereto. For oligomers, preferred examples of pharmaceutically acceptable salts include but are not limited to (a) salts formed with cations such as sodium, potassium, ammonium, magnesium, calcium, polyamines such as spermine and spermidine, etc.; (b) acid addition salts formed with inorganic acids, for example hydrochloric acid, hydrobromic acid, sulfuric acid, phosphoric acid, nitric acid and the like; (c) salts formed with organic acids such as, for example, acetic acid, oxalic acid, tartaric acid, succinic acid, maleic acid, fumaric acid, gluconic acid, citric acid, malic acid, ascorbic acid, benzoic acid, tannic acid, palmitic acid, alginic acid, polyglutamic acid, naphthalenesulfonic acid, methanesulfonic acid, p-toluenesulfonic acid, naphthalenedisulfonic acid, polygalacturonic acid, and the like; and (d) salts formed from elemental anions such as chlorine, bromine, and iodine. The pharmaceutical compositions of the present invention may be administered in a number of ways depending upon whether local or systemic treatment is desired and upon the area to be treated. Administration may be topical (including ophthalmic and mucous membranes, as well as rectal delivery), pulmonary, e.g., by inhalation or insufflation of powders or aerosols (including by nebulizer, intratracheal, intranasal, epidermal and transdermal), oral or parenteral. Parenteral administration includes intravenous, intra-arterial, subcutaneous, intraperitoneal or intramuscular injection or infusion; or intracranial, e.g., intrathecal or intraventricular, administration. Oligomers with at least one 2′-O-methoxyethyl modification are believed to be particularly useful for oral administration. Preferably, the AON is delivered via the subcutaneous or intravenous route.

The pharmaceutical formulations of the present invention, which may conveniently be presented in unit dosage form, may be prepared according to conventional techniques well known in the pharmaceutical industry. Such techniques include the step of bringing into association the active ingredients with the pharmaceutical carrier(s) or excipients(s). In general, the formulations are prepared by uniformly and intimately bringing into association the active ingredients with liquid carriers or finely divided solid carriers or both, and then, if necessary, shaping the product.

The following Examples are to be construed as merely illustrative and not limitative of the remainder of the disclosure in any way whatsoever. These Examples are included solely for the purposes of exemplifying the present invention. They should not be understood as a restriction on the broad summary, disclosure or description of the invention as set out above. Without further elaboration, it is believed that one skilled in the art can, using the preceding description, utilize the present invention to its fullest extent. In the foregoing and in the following examples, all temperatures are set forth uncorrected in degrees Celsius; and, unless otherwise indicated, all parts and percentages are by weight.

### EXAMPLES

### Examples

### Example 1—Experimental Strategy

### Background

A strategy was employed in which a selection of common variants (CVs) in the FUS gene has been selected that occur frequently in the population. For each CV, AONs can be optimised to maximise selectivity to each allele. In order to be suitable for CV targeted allele specific AON therapy, a patient would need to be heterozygous for one of the selected CVs. DNA of patients with confirmed pathogenic variants in FUS could be sequenced to determine their genotype at the selected CVs in the FUS gene. Utilising long read sequencing or other methods, the allele that their pathogenic variant falls on relative to their heterozygous CV could be determined. The appropriate CV targeted AON could then be selected for the patient which would selectively knock down the allele containing the pathogenic variant. This would mean that one of only a handful of AONs could be applicable to treat the majority of FUS-ALS or FUS-FTD patients. Those that are not heterozygous for one of the CVs could be offered an alternative FUS targeted AON treatment that knocks down both alleles equally.

### FUS Common Variant Analysis

The most common type of variant is a single nucleotide polymorphism (SNP) in which there is a single base change in the genome. CVs that are located within FUS exons rather than introns were selected to make analysis easier in cell lines used for screening (which do not contain a pathogenic FUS variant).

Common variants in FUS were analysed using data from the 1000 Genomes Project. Three CVs were present in the exonic regions of the FUS gene. A synonymous variant in exon 3 (CV1), a synonymous variant in exon 4 (CV2) and a SNP in the 3′ untranslated region of exon 15 (CV3). The rs numbers, nucleotides, minor allele frequencies and proportion of people in the dataset that are heterozygous for each variant can be seen in Table 3.

TABLE 3
Characteristics and proportion of populations heterozygous for
common FUS exonic variants from the 1000 genomes project dataset.
MAF is the minor allele frequency in the 1000 genomes dataset.
MAF (highest) is the highest minor allele frequency reported in any
population including 1000 genomes phase 3, ESP and gnomAD.

| | | | | | MAF | MAF | Het % |
|---|---|---|---|---|---|---|---|
| | RS # | Loc | Var | Type | (all) | highest | (all) |
| CV1 | rs741810 | Exon 3 | C/A | syn | 0.28 | 0.49 | 32.8 |
| CV2 | rs1052352 | Exon 4 | C/T | syn | 0.47 | 0.49 | 41.6 |
| CV3 | rs4889537 | 3′UTR | G/C | SNP | 0.28 | 0.49 | 33.2 |

The genotypes for each of the individuals in the 1000 genomes dataset was analysed using SPSS to determine the proportion of people in each population that were heterozygous in one or two of the selected FUS CVs. All populations had a higher proportion of people heterozygous for CV2 than CV1 or CV3. In European, American and Asian populations the difference was small however in African populations there was a large difference as CV1 and CV3 have a much smaller minor allele frequency. All 3 variants are in linkage disequilibrium (LD) with each other however CV1 and CV3 were in higher LD than either were with CV2. This means there is little benefit in terms of additional coverage of the population for targeting both CV1 and CV3 compared to targeting either alone. Targeting CV2 as well as either CV1 or CV3 did increase the proportion of the populations that were heterozygous for at least one variant by up to 10.6% (Table 4).

TABLE 4
Percentage of populations heterozygous for at least one
FUS CV and additional population coverage by combining
2 common variants (data from 1000 genomes phase 3).

| | | | | | | | additional | | |
|---|---|---|---|---|---|---|---|---|---|
| | | | | CV | CV | CV | coverage | | |
| | | | | 1 + | 2 + | 2 + | 1 + | 2 + | 2 + |
| | CV1 | CV2 | CV3 | 3 | 1 | 3 | 3 | 1 | 3 |
| All | 32.8 | 41.6 | 33.2 | 33.3 | 46.5 | 46.9 | 0.5 | 4.9 | 5.3 |
| Europe | 42.9 | 45.9 | 42.7 | 42.9 | 56.5 | 56.5 | 0 | 10.6 | 10.6 |
| America | 45.8 | 49.9 | 45.8 | 45.8 | 57.9 | 57.9 | 0 | 8 | 8 |
| Asia | 41.1 | 41.4 | 41.6 | 41.6 | 42.9 | 43.1 | 0.5 | 1.5 | 1.7 |
| Africa | 5.3 | 34.2 | 6.7 | 6.8 | 38.8 | 39.5 | 1.5 | 4.6 | 5.3 |

### Example 2—Design of Series One Gapmers

The first round of gapmers were designed to target each of the three FUS CVs with complete complementarity to one allele and a single mismatch for the other allele. The gapmers consisted of a 10 base DNA core flanked by wings of 5 bases on either side of 2′-O-methyl modifies RNA bases with a phosphorothioate backbone. Six AONs were designed for each CV; three that were completely complementary to the ancestral allele and three that were completely complementary to the minor allele. The three gapmers differed in the position that the CV was positioned within the DNA core, at position 2, 6 or 9 of the core from the 5′ end. AON coordinates and sequences can be seen in Table 5. A scramble control gapmer was used (SEQ ID 35: ACCUUATCCAATAGCGCCUC) as well as a gapmer targeted to another area of exon 3 to act as a control for allele selectivity (Table 6). AON nomenclature was based on that described by Mann, C .J., et al., Improved antisense oligonucleotide induced exon skipping in the mdx mouse model of muscular dystrophy. The Journal of Gene Medicine, 2002. 4(6): p. 644-654 whereby the species, gene, exon number, acceptor or donor targeting and annealing coordinates are described, where “−” indicates intronic position and “+” specifies exonic location from the splice site, as described herein. AONs were ordered from TriLink Biotechnologies, Inc (San Diego, CA, USA) or ChemGenes Corporation (Wilmington, MA, USA).

TABLE 5
Sequences and properties of FUS CV targeted AONs.

| AON | SEQ | | Target | | |
|---|---|---|---|---|---|
| ID | ID | Name | allele | AON co-ordinates | AON sequence (5′ to 3′) |
| 1 | 1 | CV1a | A | FUS H3A(+96+115) | GCCAUATCCTGAAGTGUCCG |
| 2 | 2 | CV1b | A | FUS H3A(+100+119) | UCUGGCCATATCCTGAAGUG |
| 3 | 3 | CV1c | A | FUS H3A(+103+122) | UGCUCTGGCCATATCCUGAA |
| 4 | 4 | CV1d | C | FUS H3A(+96+115) | GCCAUAGCCTGAAGTGUCCG |
| 5 | 5 | CV1e | C | FUS H3A(+100+119) | UGUGGCCATAGCCTGAAGUG |
| 6 | 6 | CV1f | C | FUS H3A(+103+122) | UGCUCTGGCCATAGCCUGAA |
| 7 | 7 | CV2a | C | FUS H4A(+88+107) | GCCAGGGTAGGAGGACUGCU |
| 8 | 8 | CV2b | C | FUS H4A(+92+111) | CAUAGCCAGGGTAGGAGGAC |
| 9 | 9 | CV2c | C | FUS H4A(+95+114) | GGCCATAGCCAGGGTAGGAG |
| 10 | 10 | Cv2d | T | FUS H4A(+88+107) | GCCAGGATAGGAGGACUGCU |
| 11 | 11 | CV2e | T | FUS H4A(+92+111) | CAUAGCCAGGATAGGAGGAC |
| 12 | 12 | CV2f | T | FUS H4A(+95+114) | GGCCATAGCCAGGATAGGAG |
| 13 | 13 | CV3a | G | FUS 15A(+797+816) | UGACCTCAAGCCCTCUGAGU |
| 14 | 14 | CV3b | G | FUS 15A(+801+820) | UCAAUGACCTCAAGCCCUCU |
| 15 | 15 | CV3c | G | FUS 15A(+804+823) | AUGUCAATGACCTCAAGCCC |
| 16 | 16 | CV3d | C | FUS 15A(+797+816) | UGACCTGAAGCCCTCUGAGU |
| 17 | 17 | CV3e | C | FUS 15A(+801+820) | UCAAUGACCTGAAGCCCUCU |
| 18 | 18 | CV3f | C | FUS 15A(+804+823) | AUGUCAATGACCTGAAGCCC |

TABLE 6
Control sequences

| | | | Target | AON co- | |
|---|---|---|---|---|---|
| AON ID | SEQ ID | Name | allele | ordinates | AON sequence (5′ to 3′) |
| 35 | 35 | CVscr | Scramble | N/A | ACCUUATCCAATAGCGCCUC |
| | | | control | | |
| 36 | 36 | nsCtrl | Both | FUS | CCACUGTAACTCTGCUGUCC |
| | | | | H3A(+62+81) | |

### Materials and Methods

### Cell Line Selection for Screening

Several human fibroblast cell lines were sequenced using sanger sequencing to determine their genotypes at the three FUS CVs. Three cell lines that differed in their genotypes at the CVs were selected for use in screening. The genotypes of each cell line at the FUS CVs can be seen in Table 7. Each cell line was transfected with the FUS CV targeted gapmers at a range of concentrations and RNA collected 24 hours after transfection. FUS RNA levels were measured using RT-PCR visualised using agarose gel electrophoresis and quantified via densiometric analysis using TBP as a housekeeping gene.

TABLE 7
Genotypes at FUS CVs of human fibroblast cell
lines used for screening CV targeted gapmers.

| Cell line | CV1 | CV2 | CV3 |
|---|---|---|---|
| Hom 1 | C/C | T/T | G/G |
| Hom 2 | A/A | C/C | C/C |
| Het | C/A | C/T | G/C |

### Transfection of Fibroblasts

Normal human dermal fibroblasts were propagated according to established techniques with 15,000 cells seeded into 24 well plates in 10% FBS DMEM and incubated at 37° C. for 24 hours prior to transfection. All AONs were transfected using Lipofectamine 3000 (3 μl per ml of transfection volume) (Life Technologies, Melbourne, Australia), according to manufacturer's protocols, and AON transfected cells incubated for 24 hours.

### Transcript Analysis

RNA was extracted using the MagMAX-96 Total RNA Isolation Kit, including a DNase treatment (Life Technologies), according to the manufacturer's instructions. RT-PCRs were performed using the One-step Superscript III RT-PCR kit with Platinum Taq polymerase (Life Technologies) according to manufacturer's instructions. Products were amplified across FUS exons 1 to 6 (SEQ ID: 37 Fwd: GTACTCAGCGGTGTTGGAAC, SEQ ID 38: Rev: CCACTACTCATGGAGGATTG), with the temperature profile, 55° C. for 30 min, 94° C. for 2 min, followed by 22 cycles of 94° C. for 30 sec, 58° C. for 30 sec and 68° C. for 1 min. Where applicable, results were normalised to transcript levels of an unrelated housekeeping control gene (TBP) amplified across exons 2 to 3 using the following primers (SEQ ID 39: Fwd: AGCGCAAGGGTTTCTGGTTT, Rev: SEQ ID 40: GGAGTCATGGGGGAGGGATA).

PCR products were fractionated on 2% agarose gels in Tris-Acetate-EDTA buffer and the images captured on gel documentation system (Vilber Lourmat, Eberhardzell, Germany). Densitometric analysis was carried out using Image J. The percentage of transcript knockdown was determined by normalisation to a housekeeping gene and comparison to control treated or untreated samples.

### Example 3—Screening of Series One CV1 Targeted Gapmers Result

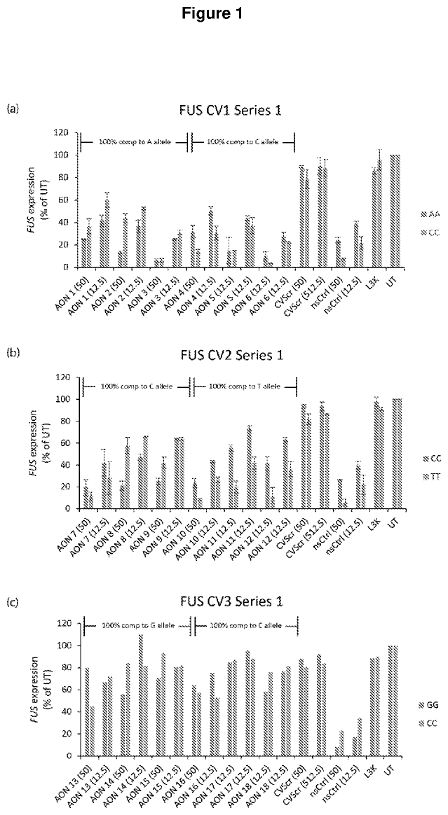

Fibroblast cell lines (Hom 1 and Hom 2) were treated with AONs targeted to CV1 (SEQ ID: 1 to 6) or control sequences including a scramble control (CVscr) (SEQ ID: 35) and a selectivity control targeted to an area in exon 3 that is identical in each allele (nsCtrl) (SEQ ID: 36) at 50 nM and 12.5 nM concentrations and collected after 24 hours in two independent experiments. Both cell lines treated with gapmers targeted to FUS CV1 showed reduced expression of FUS mRNA. All gapmers with 100% complementarity to the A allele (SEQ ID: 1 to 3) induced greater knockdown of FUS in the cell line that was homozygous A at the CV position than in the cell line that was homozygous C. Likewise all gapmers with 100% complementarity to the C allele (SEQ ID: 4 to 6) induced greater FUS knockdown in the homozygous C cell line than in the homozygous A cell line (FIG. 1a). A dose response could be seen for all CV targeted gapmers with less FUS expression when treated at 50 nM than 12.5 nM. Notably, the control gapmer that was targeted to an area of the FUS transcript without a SNP to act as a control for selectivity (nsCtrl) (SEQ ID: 36) caused greater knockdown in the homozygous C cell line than the homozygous A cell line in both experiments. This could indicate either a reduced transfection efficiency of the homozygous A cell line (Hom 2) or differences between the cell lines in the rate or efficiency of any of the cellular processes involved.

CV1b (SEQ ID: 2) showed the greatest selectivity to the A allele producing the greatest difference between FUS expression in the two homozygous cell lines at 50 nM, with the homozygous A cell line expressing 14% and the homozygous C cell line 44% of levels seen in untreated control cells. CV1a (SEQ ID: 1) also showed some selectivity to the A allele. CV1c (SEQ ID: 3) treated cells expressed similar levels of FUS in both cell lines.

AONs targeted to the C allele showed less discrimination between alleles with AONs CV1e (SEQ ID: 5) and CVIf (SEQ ID: 6) knocking down FUS to similar degrees in both cell lines. CV1d (SEQ ID: 4) showed the greatest difference in FUS expression between the cell lines at 32% of untreated control levels in the homozygous A cell line at 50 nM and at 14% in the homozygous C cell line. This may not indicate any true selectivity however as FUS levels in the nsCtrl (SEQ ID: 36) treated cells were measured at 24% and 8% of untreated control levels.

### Example 4—Screening of Series One CV2 Targeted Gapmers

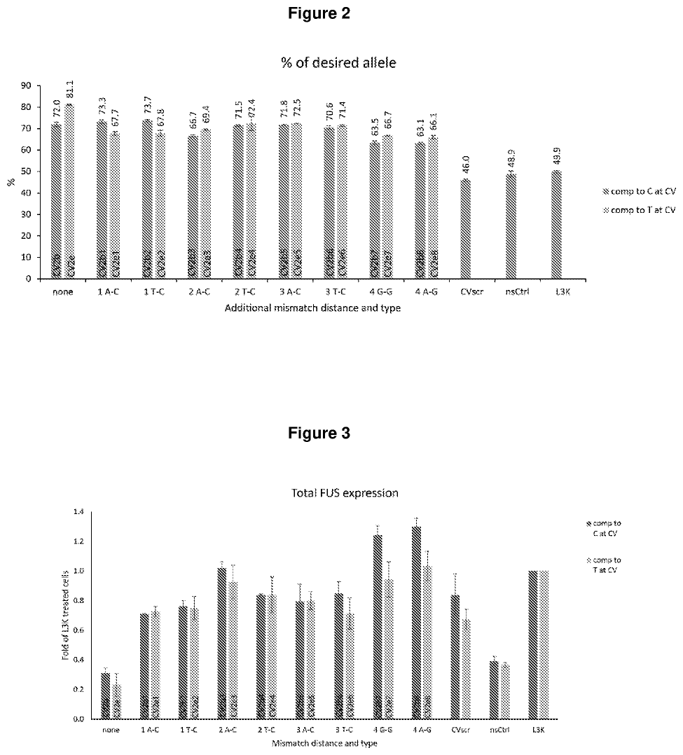

Fibroblast cell lines (Hom 1 and Hom 2) were treated with AONs targeted to CV2 (SEQ ID: 7 to 12) or control sequences (SEQ ID: 35 and 36) at 50 nM and 12.5 nM concentrations and collected after 24 hours in two independent experiments. FUS expression was analysed and can be seen in FIG. 2b. A dose response could be seen for all CV2 targeted gapmers with less FUS expression when treated at 50 nM than 12.5 nM. For gapmers with 100% complementarity to the C allele (SEQ ID: 7 to 9), CV2b (SEQ ID: 8) showed the greatest difference in FUS expression levels between homozygous cell lines in two experiments with FUS reduced to 21% of control levels at 50 nM in in the homozygous C cell line and to only 58% in the homozygous T cell line. CV2c (SEQ ID: 9) also showed some selectivity to the C allele with greater knockdown in the homozygous C cell line. In contrast cells treated with CV2a (SEQ ID: 7) produced slightly greater FUS knockdown in the homozygous T cell line. This does not indicate that there was any selectivity to the T allele however as again, there was a difference in the FUS knockdown levels in the nsCtrl treated cells between the two cell lines which needs to be taken into account when interpreting the results. Of the gapmers targeted to the T allele, CV2e (SEQ ID: 5) showed the greatest difference in expression between homozygous cell lines. The difference was only slightly lower for CV2d (SEQ ID: 10) and CV2f (SEQ ID: 12).

### Example 5—Screening of Series One CV3 Targeted Gapmers

Fibroblast cell lines (Hom 1 and Hom 2) were treated with AONs targeted to CV3 (SEQ ID: 13 to 18) or control sequences (SEQ ID: 35 and 36) at 50 nM and 12.5 nM concentrations and collected after 24 hours in two independent experiments. Gapmers targeted to CV3 did not reduce FUS expression to the same extent as was seen for CV1 and CV2 targeted AOs with some not showing knockdown greater than that seen in cells treated with the scramble control. Expression was knocked down substantially and to similar levels as in previous experiments for the control oligo targeted to exon 3 (nsCtrl) (SEQ ID: 36) indicating that there this wasn't due to poor transfection efficiency. There was no obvious dose response seen across most treatments with the exception of the nsCtrl oligo (SEQ ID: 36). Furthermore, there was no trend toward expression levels being lowered in the homozygous cell line with 100% complementarity to the transfected gapmer as was seen for CV1 and CV2.

### Example 6—Design of CV2 Series 2 Gapmers With an Additional Mismatch

A second series of gapmers was designed to target CV2. The data from the first series of gapmers showed that a centrally located mismatch improved the selectivity between alleles. CV2b (SEQ ID: 8) was selected to target the C allele, and CV2e (SEQ ID: 11) to target the T allele with the second series of gapmers designed around these sequences with an extra mismatch added. These two AONs are identical apart from the single base change that is located in the centre of the DNA gap of the molecule at position 6 out of 10 from the 5′ end.

### Adding an Additional Mismatch

A mismatched base pair is one that does not conform to Watson Crick base pairing rules (guanine-cytosine and adenine-thymine). Mismatches are defined as a transduction when they are formed by non-complementary pyrimidine/purine bases. Those that are formed by a purine/purine or pyrimidine/pyrimidine pair are transversions (Modrich, P., DNA MISMATCH CORRECTION. Annual Review of Biochemistry, 1987. 56(1): p. 435-466). Mismatches can produce major alterations in the structure and stability of nucleic acid duplexes, especially in the case of purine transversions (Rossetti, G., et al., The structural impact of DNA mismatches. Nucleic Acids Research, 2015. 43(8): p. 4309-4321). DNA usually exists in a double stranded duplex held together by hydrogen bonds in the base pairing model described by Watson and Crick, although this is the most stable structure, fluctuations do occur. DNA breathing refers to the transient opening of nucleic acid base pairs due to conformational or thermal fluctuations below the melting temperature of the duplex. This results in the formation of transient structures with lower stability. This is an important mechanism which allows polymerases and other DNA regulatory proteins access to DNA coding templates that are located within the interior of the DNA duplex (Phelps, C., et al., Single-molecule FRET and linear dichroism studies of DNA breathing and helicase binding at replication fork junctions. Proceedings of the National Academy of Sciences of the United States of America, 2013. 110(43): p. 17320-17325). Using NMR spectroscopy and molecular dynamics simulations, the structural alterations and frequency of breathing has been characterised for the canonical base pairs and each of the DNA mismatches. The breathing propensity of the canonical base pairs is relatively low has been measured at <0.2% for C/G and <3% for A/T. This can vary greatly for mismatched base pairs with some such as C/C having a low breathing propensity of around 1%. G/G and A/A in contrast have breathing propensities >40% with the other mismatches somewhere in between (see Rossetti et al.).

In some cases, a single mismatch in a DNA/RNA heteroduplex can result in a several fold decrease in cleavage by RNase H (Giles, R. V., et al., Single base discrimination for ribonuclease H-dependent antisense effects within intact human leukaemia cells. Nucleic Acids Research, 1995. 23(6): p. 954-961). Single mismatch discrimination of cleavage by RNase H is highly dependent on the nature of the SNP and the surrounding nucleotides. The addition of extra mismatches in gapmers designed to target a specific allele can in some cases increase differentiation between two alleles. In other cases this does not occur and the opposite effect can even be seen (Magner, D., et al., Influence of mismatched and bulged nucleotides on SNP-preferential RNase H cleavage of RNA-antisense gapmer heteroduplexes. Scientific Reports, 2017. 7(1): p. 12532). The position of the mismatch within the DNA window and in relation to the position of another mismatch may also influence RNase H activity.

Eight new AONs were designed to target each FUS allele (SEQ ID: 19 to 34) with CV2 remaining in the same position and extra mismatches added. Mismatches were added at varying locations (1, 2, 3 or 4 bases away from CV2) and with different nucleotides selected for the mismatch based on the amount of breathing that would be allowed (high or low). The AON sequences and properties can be seen in Table 8.

TABLE 8
Sequences and properties of FUS CV2 targeted AONs-Series 2.

| | | | | | | Dist | | |
|---|---|---|---|---|---|---|---|---|
| AON | SEQ | | Target | AON co- | AON sequence | from | | Mismatch |
| ID | ID | Name | allele | ordinates | (5′ to 3′) | MM | MM | type |
| 19 | 19 | CV2b1 | C | FUS | CAUAGCCAGAGTAGGAGGAC | 1 base | A/C | transduction |
| | | | | H4A(+92+111)A1 | | | | |
| 20 | 20 | CV2b2 | C | FUS | CAUAGCCAGTGTAGGAGGAC | 1 base | T/C | Pyrimidine |
| | | | | H4A(+92+111)A1 | | | | transversion |
| 21 | 21 | CV2b3 | C | FUS | CAUAGCCAAGGTAGGAGGAC | 2 bases | A/C | transduction |
| | | | | H4A(+92+111)A1 | | | | |
| 22 | 22 | CV2b4 | C | FUS | CAUAGCCATGGTAGGAGGAC | 2 bases | T/C | pyrimidine |
| | | | | H4A(+92+111)A1 | | | | transversion |
| 23 | 23 | CV2b5 | C | FUS | CAUAGCCAGGGTAAGAGGAC | 3 bases | A/C | transduction |
| | | | | H4A(+92+111)A1 | | | | |
| 24 | 24 | CV2b6 | C | FUS | CAUAGCCAGGGTATGAGGAC | 3 bases | T/C | pyrimidine |
| | | | | H4A(+92+111)A1 | | | | transversion |
| 25 | 25 | CV2b7 | C | FUS | CAUAGCGAGGGTAGGAGGAC | 4 bases | G/G | purine |
| | | | | H4A(+92+111)A1 | | | | transversion |
| 26 | 26 | CV2b8 | C | FUS | CAUAGCAAGGGTAGGAGGAC | 4 bases | G/A | purine |
| | | | | H4A(+92+111)A1 | | | | transversion |
| 27 | 27 | CV2e1 | T | FUS | CAUAGCCAGAATAGGAGGAC | 1 base | A/C | transduction |
| | | | | H4A(+92+111)A2 | | | | |
| 28 | 28 | CV2e2 | T | FUS | CAUAGCCAGTATAGGAGGAC | 1 base | T/C | Pyrimidine |
| | | | | H4A(+92+111)A2 | | | | transversion |
| 29 | 29 | CV2e3 | T | FUS | CAUAGCCAAGATAGGAGGAC | 2 bases | A/C | transduction |
| | | | | H4A(+92+111)A2 | | | | |
| 30 | 30 | CV2e4 | T | FUS | CAUAGCCATGATAGGAGGAC | 2 bases | T/C | pyrimidine |
| | | | | H4A(+92+111)A2 | | | | transversion |
| 31 | 31 | CV2e5 | T | FUS | CAUAGCCAGGATAAGAGGAC | 3 bases | A/C | transduction |
| | | | | H4A(+92+111)A2 | | | | |
| 32 | 32 | CV2e6 | T | FUS | CAUAGCCAGGATATGAGGAC | 3 bases | T/C | pyrimidine |
| | | | | H4A(+92+111)A2 | | | | transversion |
| 33 | 33 | CV2e7 | T | FUS | CAUAGCGAGGATAGGAGGAC | 4 bases | G/G | purine |
| | | | | H4A(+92+111)A2 | | | | transversion |
| 34 | 34 | CV2e8 | T | FUS | CAUAGCAAGGATAGGAGGAC | 4 bases | G/A | purine |
| | | | | H4A(+92+111)A2 | | | | transversion |

### Example 7—Screening of Series Two CV2 Targeted Gapmers

A human fibroblast cell line that is heterozygous at CV2 was used for screening. Using a subset of AONs, it was determined that the concentration and timepoint that gave the greatest selectivity was 24 hours after transfection at a 100 nM concentration. The proportion of each allele present in treated cells was quantified in multiple experiments at this concentration and timepoint using droplet digital PCR. Droplet digital PCR analysis was carried out using the BioRad QX200 system and the One-Step RT-ddPCR Advanced Kit for Probes. Primers amplified FUS with the Forward primer in exon 4 and the reverse primer crossing the exon ⅘ junction (SEQ ID 41: Fwd: CTATGGAACTCAGTCAACTCC, SEQ ID 42: Rev: GAACTGCTACCGTAACTTCC) with the temperature profile, 50° C. for 50 min, 95° C. for 10 min, followed by 40 cycles of 95° C. for 30 sec and 55° C. for 1 min and finally 98° C. for 10 min. Quantification of transcripts containing a T at CV2 were detected using a probe with a Hex fluorophore and lowa Black quencher (Probe 1: CCTCCT+AT+C+CTGGCT). Quantification of transcripts containing a C at CV2 were detected using a probe with a FAM fluorophore and lowa Black quencher (Probe 2: CTCCT+AC+C+CTGGCTA). Probes were purchased from Integrated DNA Technologies. Controls were run with RNA from cell lines homozygous for each allele at FUS CV2 to ensure there was no signal from the other allele for each probe.

In cells treated with AON CV2b (SEQ ID: 8) for 24 hours (100% complementarity to the C allele and 1 mismatch to the T allele) 72% of FUS transcripts were of the allele containing the T at the CV (FIG. 2). The proportion of the T allele present in CV2b1 and CV2b2 (SEQ ID: 19 and 20) treated cells was only slightly higher at 73.3 and 73.7%. Some of the AOs with additional mismatches (CV2b3, CV2b7 and CV2b8) (SEQ ID: 21, 25, 26) resulted in a significant decrease in allele selective knockdown compared to SEQ ID 8 treated cells with the T allele measured at 66.7, 63.5 and 63.1% of transcripts.

In cells treated with AON CV2e (SEQ ID: 11) (100% complementarity to the T allele and 1 mismatch to the C allele) 81.1% of FUS transcripts were of the C allele (FIG. 2). All of the additional mismatches led to a reduction in the proportion of C allele transcripts ranging from 66.1% for with CV2e8 (SEQ ID: 34) to 72.5% for CV2e5 (SEQ ID: 31).

Total FUS expression was quantified after 24 hours incubation at 100 nM using RT-PCR with agarose gel electrophoresis followed by densitometric analysis (FIG. 3). For the AONs with complete complementarity to their target allele and only 1 mismatch from the non-target allele, (SEQ ID: 8 and 11) total FUS levels were at 31% and 23% of the level seen in control treated cells (treated with transfection reagent with no AON present). When an additional mismatch was added total FUS levels ranged from 71% to 130% of control levels. As FUS is autoregulated by its protein level, there may be upregulation in response to the AON mediated knockdown.

### Example 8—CV2 Targeted AON Chemistry Comparison

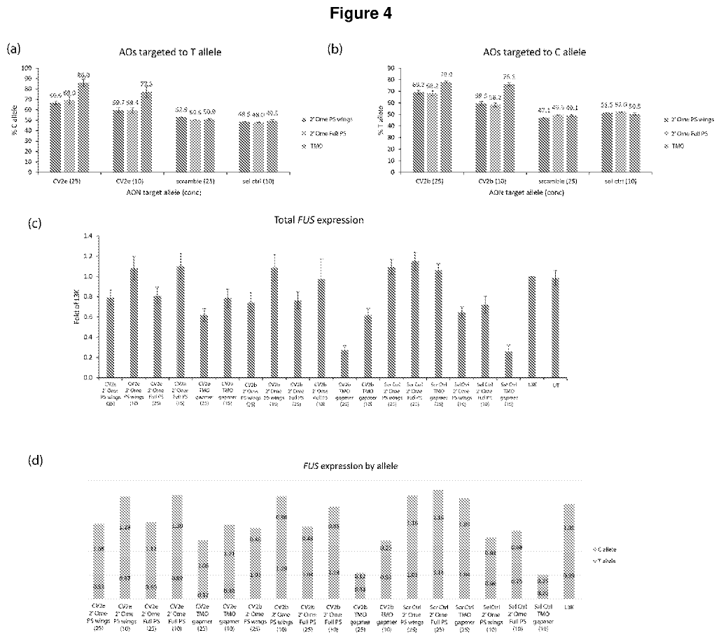

CV2 targeted AONs CV2b and CV2e were synthesised as a 2′ O-methyl gapmer with the PS modification throughout and as a thiophosphoramidate morpholino oligomer (TMO) chimera (gapmer): 5 base morpholino 3′-thiophosphoramidates wings a 10 base 2′-deoxynucleoside 3′-thiophosphates gap with a 2′-deoxynucleoside base at the 3′ end and were tested in 3 experiments in a human fibroblasts cell line that is heterozygous for CV2. Cells were transfected using lipofectamine 3000 at 25 nM and10 nM concentrations. Cells were collected 3 days following transfection and FUS transcripts analysed by ddPCR and RT-PCR and agarose gel electrophoresis. In cells transfected with CV2e (TMO gapmer) that is targeted to reduce expression of the T allele at 25 nM, 86% of transcripts after 3 days were of the C allele (FIG. 4a). In cells transfected with CV2b (TMO gapmer) that is targeted to reduce expression of the C allele at 25 nM, 78% of transcripts after 3 days were of the T allele (FIG. 4b). The proportion of transcripts of the desired allele were significantly lower when cells were transfected with the 2′-O-methyl gapmer AONs. Total FUS expression after 3 days treatment with AON CV2e at 25 nM was at 62% of control levels (FIG. 4c) indicating that overall expression of the C allele had increased slightly compared to levels in L3K treated control cells while expression of the T allele had reduced to approximately 17% of levels in controls (FIG. 4d). Total FUS expression after 3 days treatment with AON CV2b at 10 nM was at 61% of control levels (FIG. 4c) indicating that overall expression of the T allele had reduced only slightly to 93% of L3K treated control cells while expression of the C allele had reduced to approximately 29% of levels in controls (FIG. 4d).

### Sequence Listing

Due to limitations with WIPO's sequence listing software, which prevents applicants from entering sequences that contain both uracil bases and thymine bases (such as many of the sequences of the instant invention), all uracil bases in the sequences were converted to thymine bases to enable sequence listing and entry into WIPO's sequence listing software, and the molecule type was classified as “DNA”. However, where there is a difference in information between a sequence in the body of this specification and a sequence listed in the sequence listing file, the body of this specification is the master reference for the sequence and information regarding the invention.

1. An antisense oligonucleotide targeted to a nucleic acid molecule encoding FUS pre-mRNA or mRNA, wherein the antisense oligonucleotide comprises a nucleotide sequence
a) a nucleotide sequence comprising any one of SEQ ID NOs: 1-34, or a variant thereof; or
b) a nucleotide sequence complementary to at least 1 or more contiguous nucleobases in a target FUS pre-mRNA or mRNA to which any one of the nucleotide sequences of SEQ ID NOs: 1-34, or a variant thereof also binds,
wherein the antisense oligonucleotide inhibits the expression of at least one allele of the FUS gene or a FUS gene variant thereof, and
wherein the antisense oligonucleotide is substantially isolated or purified.

2. The antisense oligonucleotide of claim 1, wherein the allele comprises a pathogenic variant of the FUS gene.

3. The antisense oligonucleotide of claim 2, wherein the pathogenic variant is a genetic alteration that increases the subject's susceptibility or predisposition to a disease.

4. The antisense oligonucleotide of claim 3, wherein the disease is selected from the group consisting of: ALS, FTD, ET.

5. The antisense oligonucleotide of claim 4, wherein the disease is selected from the group consisting of: ALS or FTD.

6. The antisense oligonucleotide of claim 5, wherein the disease is selected from the group consisting of: FUS-ALS or FUS-FTD.

7. The antisense oligonucleotide of claim 1 that binds to an area of the transcript comprised within exon 3, exon 4 or 3′UTR on FUS.

8. The antisense oligonucleotide of claim 7, wherein the antisense oligonucleotide binds to an area of the transcript comprising CV (rs741810), CV2 (rs1052352), or CV3 (rs4889537).

9. The antisense oligonucleotide of claim 1 that selectively knocks down expression of one allele of the pre-mRNA and/or mRNA of FUS or a FUS variant thereof whilst leaving enough of the normal mRNA present to produce a substantial amount of protein.

10. The antisense oligonucleotide of claim 1 that induces RNase H mediated degradation of the FUS pre-mRNA and/or FUS mRNA.

11. The antisense oligonucleotide of claim 1 that is a thiophosphoramidate morpholino oligomer (TMO) chimera.

12. The antisense oligonucleotide of claim 11 wherein the thiophosphoramidate morpholino oligomer (TMO) chimera has a gapmer design comprising TMO and phosphorothioate DNA or DNA subunits.

13. The antisense oligonucleotide of claim 11 that is a peptide-thiophosphoramidate morpholino oligomer (TMO) chimera conjugate.

14. The antisense oligonucleotide of claim 1 that comprises a nucleotide sequence of any one of SEQ ID NOs: 1 to 12.

15. The antisense oligonucleotide of claim 14 that comprises the nucleotide sequence of SEQ ID NO: 2, 4, 5, 8, or 11.

16. A method of inducing selective knockdown of a selected allele of FUS pre-mRNA and/or mRNA, the method comprising the steps of:
a) providing the antisense oligonucleotide of claim 1; and
b) allowing the antisense oligonucleotide to bind to a target nucleic acid site on the pre-mRNA and/or RNA.

17. (canceled)

18. A composition comprising:
a) the antisense oligonucleotide of claim 1; and
b) a pharmaceutically acceptable carrier and/or diluent.

19. A method of treating, preventing or ameliorating the effects of a disease associated with a pathogenic variant of a FUS gene in a subject, the method comprising the step of administering to the subject an effective amount of the pharmaceutical composition of claim 18.

20. The method of claim 19, wherein the pathogenic variant is a genetic alteration that increases the subject's susceptibility or predisposition to the disease.

21. The method of claim 20, wherein the disease is ALS, FTD or ET.

22. The method of claim 21, wherein the disease is ALS or FTD.

23. The method of claim 22, wherein the disease is FUS-ALS or FUS-FTD

24. A method for treating, preventing or ameliorating the effects of a disease associated with a pathogenic variant in a FUS gene in patients identified by a biomarker, the method comprising the step of:
a) testing a subject for the presence of a biomarker associated with a disease associated with FUS proteinopathy where patients are likely to respond to FUS suppression; and
b) if the subject is found to express the biomarker, administering to the subject an effective amount of the composition of claim 18.

25. The method of claim 24, wherein the disease is selected from the group consisting of: ALS or FTD.

26. The method of claim 24, wherein the biomarker is a pathogenic variant of the FUS gene.

27. A method of reducing the expression of selected FUS alleles in a subject and/or reducing the over expression of FUS caused by auto regulation in a subject, the method comprising the step of administering to the subject an effective amount of the composition of claim 18.

28. A method of reducing the expression of selected FUS alleles in a subject; and/or reducing the overexpression of FUS caused by autoregulation in a subject, the method comprising the step of administering to the subject an effective amount of
the composition of claim 18.

29. An expression vector comprising the antisense oligonucleotide of claim 1.

30. A cell comprising the antisense oligonucleotide of claim 1.

31.-33. (canceled)

34. A kit comprising the antisense oligonucleotide of claim 1 packaged in a suitable container, optionally including instructions for use of the oligonucleotide.
